(19) United States
(12) Patent Application Publication
(10) Pub. No.: US 2022/0062529 A1
(43) Pub. Date: Mar. 3, 2022

(54) NEGATIVE PRESSURE WOUND THERAPY SYSTEM

(71) Applicant: KCI LICENSING, INC., San Antonio, TX (US)

(72) Inventors: Benjamin A. PRATT, Poole (GB); Christopher A. CARROLL, San Antonio, TX (US); Justin RICE, San Antonio, TX (US); James SEDDON, Ferndown (GB)

(21) Appl. No.: 17/419,566

(22) PCT Filed: Dec. 27, 2019
(86) PCT No.: PCT/US2019/068772

§ 371 Date: Jun. 29, 2021

Related U.S. Application Data
(60) Provisional application No. 62/791,194, filed on Jan. 11, 2019.

(51) Int. Cl. A61M 1/00; A61F 13/02

(52) U.S. Cl. CPC A61M 1/912; A61M 1/985; A61F 13/0216; A61M 2205/585; A61M 1/92; A61M 1/95; A61F 13/0206

(57) ABSTRACT

A releasable connector for a negative pressure wound therapy system having a drape and a tube includes a base and a tube fitting. The base is configured to be coupled to the drape such that a substantially air-tight seal is formed between the base and the drape. The base includes a base locking member. The tube fitting is configured to be coupled to the tube such that a substantially air-tight seal is formed between the tube fitting and the tube. The tube fitting includes a tube locking member. The tube fitting is further configured to be selectively coupled to the base through an interaction between the tube locking member and the base locking member such that a substantially air-tight seal is formed between the tube fitting and the base.

### CROSS-REFERENCE TO RELATED APPLICATIONS

This application claims the benefit of priority to U.S. Provisional Application No. 62/791,194, filed on Jan. 11, 2019, which is incorporated herein by reference in its entirety.

### BACKGROUND

The present disclosure relates generally to wound therapy systems and devices, and more particularly to a negative pressure wound therapy system with a releasable connector.

Negative pressure wound therapy (NPWT) is a type of wound therapy that involves applying negative pressure (relative to atmospheric pressure) to a wound site to promote wound healing. Some NPWT systems surround the wound in a dressing which is sealed with a drape. The drape establishes a barrier between negative pressure and atmospheric pressure. These NPWT systems may include a port that, when installed on a drape, connects the NPWT system to the wound underneath the dressing and the drape. In order to access the wound after the port has been installed, the dressing must typically be removed. As a result, a new dressing must be reinstalled so that NPWT can continue, thereby leading to excessive dressing changes. It would be desirable to provide a dressing having an in-situ wound interaction and observation capability.

### SUMMARY

One implementation of the present disclosure is a releasable connector for a negative pressure wound therapy system having a drape and a tube. The releasable connector includes a base and a tube fitting. The base is configured to be coupled to the drape such that a substantially air-tight seal is formed between the base and the drape. The base includes a base locking member. The tube fitting is configured to be coupled to the tube such that a substantially air-tight seal is formed between the tube fitting and the tube. The tube fitting includes a tube locking member. The tube fitting is further configured to be selectively coupled to the base through an interaction between the tube locking member and the base locking member such that a substantially air-tight seal is formed between the tube fitting and the base.

In some embodiments, the interaction between the tube locking member and the base locking member is a rotation of the tube fitting relative to the base.

In some embodiments, the interaction between the tube locking member and the base locking member is a lateral translation of the tube fitting relative to the base.

In some embodiments, the interaction between the tube locking member and the base locking member is an axial translation of the tube fitting relative to the base.

In some embodiments, the releasable connector further includes a lens fitting. The lens fitting includes a magnifying lens and a lens fitting locking member configured to be selectively coupled to the base to permit examination of a wound bed. In some embodiments, the interaction between the lens fitting locking member and the base locking member is one of: a rotation of the lens fitting relative to the base; a lateral translation of the lens fitting relative to the base; or an axial translation of the lens fitting relative to the base. In some embodiments, the base is configured to be coupled to only one of the lens fitting and the tube fitting at any one time.

In some embodiments, the releasable connector further includes a removable and replaceable absorbent core accessible through the base. In some embodiments, the absorbent core is configured to be removable through the base to permit access to a wound bed.

In some embodiments, the releasable connector further includes a manual instillation connection fitting that is configured to be selectively coupled to the base to permit manual instillation of a fluid to a wound bed or analysis of wound exudate from a wound bed. In some embodiments, the base is configured to be coupled to only one of the manual instillation connection fitting and the tube fitting at any one time.

Another implementation of the present disclosure is a method of accessing a wound bed through an in-situ negative pressure wound therapy dressing. The method includes disconnecting a tube fitting from a base to expose the in-situ negative pressure wound therapy dressing. The method also includes removing a portion of the in-situ negative pressure wound therapy dressing through the base. The method also includes instituting a treatment regimen through the base.

In some embodiments, the treatment regimen includes at least one of: conducting a biopsy; conducting an inspection of the wound bed; manually instilling a fluid to the wound bed; obtaining a wound exudate sample; or installing a new tube.

In some embodiments, the method further includes, after the portion of the in-situ negative pressure wound therapy dressing has been removed through the base and before the treatment regimen has been instituted through the base, coupling a lens to the base. The treatment regimen includes conducting an inspection of a wound bed through the lens.

In some embodiments, the method further includes, after the portion of the in-situ negative pressure wound therapy dressing has been removed through the base and after the treatment regimen has been instituted through the base, reconnecting the tube fitting to the base to cover the in-situ negative pressure wound therapy dressing such that a substantially air-tight seal is formed between the tube fitting and the base. In some embodiments, reconnecting the tube fitting to the base includes rotating the tube fitting relative to the base and the substantially air-tight seal is formed through rotation of the tube fitting relative to the base. In some embodiments, reconnecting the tube fitting to the base includes laterally translating the tube fitting relative to the base and the substantially air-tight seal is formed through lateral translation of the tube fitting relative to the base. In some embodiments, reconnecting the tube fitting to the base includes axially translating the tube fitting relative to the base and the substantially air-tight seal is formed through axial translation of the tube fitting relative to the base.

In some embodiments, the method further includes, after the treatment regimen has been instituted, inserting a removable and replaceable absorbent core through the base. In some embodiments, the method further includes, after the removable and replaceable absorbent core has been inserted through the base, reconnecting the tube fitting to the base to cover the in-situ negative pressure wound therapy dressing.

In some embodiments, the method further includes, after the portion of the in-situ negative pressure wound therapy dressing has been removed through the base, connecting a manual instillation connection fitting to the base.

Another implementation of the present disclosure is a negative pressure wound therapy system. The negative pressure wound therapy system includes a dressing, a drape, a connector base, a tube fitting, a tube, and a negative pressure device. The dressing includes a foam material. The drape has an opening overlaying the foam material. The connector base is coupled to the drape about the opening. The connector base has one of a receptacle and a projection. The tube fitting has the other of the receptacle and the projection. The tube fitting is releasably engageable with the connector base via a coupling interaction between the receptacle and the projection. The tube is coupled to the tube fitting. The negative pressure device is coupled to the tube.

In some embodiments, the coupling interaction between the receptacle and the projection creates a substantially air-tight seal between the connector base and the tube fitting.

In some embodiments, the coupling interaction is one of: a rotation of the tube fitting relative to the connector base; a linear translation of the tube fitting relative to the connector base; and an axial translation of the tube fitting relative to the connector base.

In some embodiments, the negative pressure wound therapy system further includes a removable and replaceable absorbent core configured to be received within the connector base.

In some embodiments, the negative pressure wound therapy system further includes a lens fitting that includes a magnifying lens. The lens fitting is configured to be selectively coupled to the connector base to facilitate examination of a wound bed. In some embodiments, the connector base is configured to be coupled to only one of the lens fitting and the tube fitting at any one time.

In some embodiments, the negative pressure wound therapy system further includes a manual instillation connection fitting that is configured to be selectively coupled to the connector base to permit manual instillation of a fluid to a wound bed or analysis of wound exudate from a wound bed. In some embodiments, the connector base is configured to be coupled to only one of the manual instillation connection fitting and the tube fitting at any one time.

### BRIEF DESCRIPTION OF THE DRAWINGS

FIG. 1 is a drawing of a negative pressure wound therapy (NPWT) system including a NPWT device fluidly connected with a wound site, according to an exemplary embodiment.

FIG. 2 is a cross-sectional view of a releasable connector coupled to a drape, which is covering a dressing that covers a wound bed, according to an exemplary embodiment.

FIG. 3 is an exploded view of a releasable connector, according to an exemplary embodiment.

FIG. 4 is a perspective view of a fitting for the releasable connector shown in FIG. 3, according to an exemplary embodiment.

FIG. 5 is a perspective view of another fitting for the releasable connector shown in FIG. 3, according to an exemplary embodiment.

FIG. 6 is a perspective view of yet another fitting for the releasable connector shown in FIG. 3, according to an exemplary embodiment.

FIG. 7 is an exploded view of a releasable connector, according to an exemplary embodiment.

FIG. 8 is a perspective view of a fitting for the releasable connector shown in FIG. 7, according to an exemplary embodiment.

FIG. 9 is a perspective view of another fitting for the releasable connector shown in FIG. 7, according to an exemplary embodiment.

FIG. 10 is a perspective view of yet another fitting for the releasable connector shown in FIG. 7, according to an exemplary embodiment.

FIG. 11 is an exploded view of a releasable connector, according to an exemplary embodiment.

FIG. 12 is a perspective view of a fitting for the releasable connector shown in FIG. 11, according to an exemplary embodiment.

FIG. 13 is a perspective view of another fitting for the releasable connector shown in FIG. 11, according to an exemplary embodiment.

FIG. 14 is a perspective view of yet another fitting for the releasable connector shown in FIG. 11, according to an exemplary embodiment.

FIG. 15 is an exploded view of a releasable connector, according to an exemplary embodiment.

FIG. 16 is a perspective view of a fitting for the releasable connector shown in FIG. 15, according to an exemplary embodiment.

FIG. 17 is a perspective view of another fitting for the releasable connector shown in FIG. 15, according to an exemplary embodiment.

FIG. 18 is a perspective view of yet another fitting for the releasable connector shown in FIG. 15, according to an exemplary embodiment.

FIG. 19 is an exploded view of a releasable connector, according to an exemplary embodiment.

FIG. 20 is a perspective view of a fitting for the releasable connector shown in FIG. 20, according to an exemplary embodiment.

FIG. 21 is a perspective view of another fitting for the releasable connector shown in FIG. 20, according to an exemplary embodiment.

FIG. 22 is a perspective view of yet another fitting for the releasable connector shown in FIG. 20, according to an exemplary embodiment.

FIG. 23 is an exploded view of a releasable connector, according to an exemplary embodiment.

FIG. 24 is a perspective view of a fitting for the releasable connector shown in FIG. 23, according to an exemplary embodiment.

FIG. 25 is a perspective view of another fitting for the releasable connector shown in FIG. 23, according to an exemplary embodiment.

FIG. 26 is a perspective view of yet another fitting for the releasable connector shown in FIG. 23, according to an exemplary embodiment.

FIG. 27 is an exploded view of a releasable connector, according to an exemplary embodiment.

FIG. 28 is a perspective view of a fitting for the releasable connector shown in FIG. 27, according to an exemplary embodiment.

FIG. 29 is a perspective view of another fitting for the releasable connector shown in FIG. 27, according to an exemplary embodiment.

FIG. 30 is a perspective view of yet another fitting for the releasable connector shown in FIG. 27, according to an exemplary embodiment.

FIG. 31 is an exploded view of a releasable connector, according to an exemplary embodiment.

FIG. 32 is a perspective view of a fitting for the releasable connector shown in FIG. 31, according to an exemplary embodiment.

FIG. 33 is a perspective view of another fitting for the releasable connector shown in FIG. 31, according to an exemplary embodiment.

FIG. 34 is a perspective view of yet another fitting for the releasable connector shown in FIG. 31, according to an exemplary embodiment.

FIG. 35 is an exploded view of a releasable connector, according to an exemplary embodiment.

FIG. 36 is a perspective view of a fitting for the releasable connector shown in FIG. 35, according to an exemplary embodiment.

FIG. 37 is a perspective view of another fitting for the releasable connector shown in FIG. 35, according to an exemplary embodiment.

FIG. 38 is a perspective view of yet another fitting for the releasable connector shown in FIG. 35, according to an exemplary embodiment.

FIG. 39 is a drawing of a kit for use with a NPWT device, according to an exemplary embodiment.

FIG. 40 is a block diagram of a method of using a NPWT device with a releasable connector according to an exemplary embodiment.

FIG. 41 is a block diagram of a method of using a NPWT device with a releasable connector according to an exemplary embodiment.

### DETAILED DESCRIPTION

### Overview

Referring generally to the FIGURES, a releasable connector for use with a negative pressure wound therapy (NPWT) system is shown, according to various exemplary embodiments. The releasable connector includes a connector base and a fitting. The connector base is configured to be coupled to a NPWT drape. The NPWT drape may be covering a dressing which may be covering or may at least partially surround a tissue wound. The connector base is configured to facilitate fluid communication through the drape while maintaining a substantially air-tight seal between the drape and the connector base.

The fitting is configured to be selectively coupled to, and selectively decoupled from, the connector base. When the fitting is coupled to the connector base, an NPWT device of the NPWT may perform NPWT by drawing air and/or wound exudate out from under the drape via the connecting base and the fitting. When the fitting is not coupled to the connector base, access through the connector base may be facilitating. This access can facilitate, for example, inspection of a tissue wound, manual instillation, biopsy retrieval, or insertion of a removable manifold core.

To facilitate coupling between the connector base and the fitting, the connector base and the fitting each include complimentary coupling members. In some embodiments, the connector base or the fitting includes a projection and the other of the connector base and the fitting includes a receptacle configured to receive the projection.

Throughout NPWT, it is typically desired to utilize access through the NPWT drape. Typically, this involves cutting the NPWT drape and, after access through the NPWT drape is no longer needed, applying a new NPWT drape. This leads to excessive use of NPWT drapes and is inefficient. The NPWT system described herein facilitates access through an NPWT drape without requiring cutting of the NPWT to facilitate such access. Instead, the NPWT system described here utilizes a releasable connector which includes a fitting and a connector base which can be selectively coupled and decoupled to facilitate access through the NPWT. In this way, the NPWT system described herein reduces NPWT drape consumption and increases efficiency. These and other features and advantages of the NPWT system are described in detail below.

### NPWT System

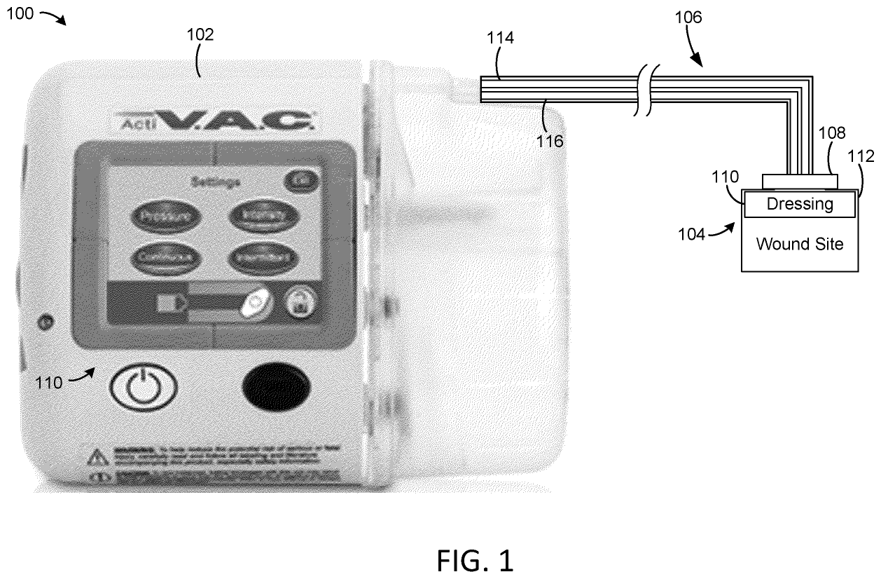

Referring now to FIG. 1, a NPWT system 100 is shown, according to an exemplary embodiment. NPWT system 100 is shown to include a therapy device 102 fluidly connected to a wound site 104 via tubing 106 and a reduced pressure interface (shown as a port 108). Wound site 104 include a tissue wound, a dressing 110 that covers the tissue wound and adheres to a patient's skin, and a drape 112 that covers dressing 110 and port 108. Drape 112 may adhere to port 108, dressing 110, and/or the patient's skin. Drape 112 facilitates a pressure differential between wound site 104 and a surrounding atmosphere. Dressing 110 may be referred to as being a dressing layer and drape 112 may be referred to as a drape layer.

Therapy device 102 can be configured to provide negative pressure wound therapy by reducing the pressure at wound site 104. Therapy device 102 can draw a vacuum at wound site 104 (relative to atmospheric pressure) by removing wound exudate, air, and other fluids from wound site 104 through port 108 and tubing 106. Wound exudate may include fluid that filters from a patient's circulatory system into lesions or areas of inflammation. For example, wound exudate may include water and dissolved solutes such as blood, plasma proteins, white blood cells, platelets, and red blood cells. Other fluids removed from wound site 104 may include instillation fluid previously delivered to wound site 104. Instillation fluid can include, for example, a cleansing fluid, a prescribed fluid, a medicated fluid, an antibiotic fluid, or any other type of fluid which can be delivered to wound site 104 during wound treatment. The fluids removed from wound site 104 pass through port 108 and then through tubing 106 and may be collected in a canister that is configured to collect wound exudate and other fluids removed from wound site 104. In addition to providing removing exudate, air, and other fluids from wound site 104, tubing 106 may include separate lumens for use by therapy device 102 to measure pressure within wound site 104.

In various embodiments, tubing 106 is a multi-lumen tubing having at least a first lumen 114 and a second lumen 116. First lumen 114 is configured to convey pressure from therapy device 102 to wound site 104 and second lumen 116 is configured to convey wound exudate and other fluids from wound site 104 to therapy device 102 or to a canister for collection. Tubing 106 may also include additional lumens (e.g., another lumen like first lumen 114, another lumen like second lumen 116, etc.) in some embodiments.

### Set Up of a Typical System

When a typical system is set up, a dressing is applied over a wound site and the dressing is covered by a drape. A port is installed by attaching the port to the drape. For example, the port may be adhesively affixed to the drape so as to create a substantially air tight seal between the drape and the port. A hole may be made in the drape so as to facilitate fluid communication with the wound site through the port. Once the port is installed, a therapy device can be used to perform NPWT on the wound site.

If it is desired to access the wound site, such as to remove a manifold from the port, to inspect the wound site, to obtain a sample from the wound site, to perform instillation (e.g., manual instillation, etc.), or to perform analysis of exudate from the wound site, the port must typically be removed (e.g., uninstalled, etc.). In many applications, the port must be cut from the drape in order for the port to be removed. After access to the wound site is no longer desired (e.g., after a manifold has been removed from the port, after the wound site has been inspected, after a sample has been obtained from the wound site, after instillation has been performed, after analysis of exudate from the wound site has been performed, etc.), and it is instead desired to again perform NPWT on the wound site, the port must typically be reinstalled (e.g., a new drape must be used to cover the dressing, etc.). Accordingly, access to a wound site after a port has been installed (e.g., after NPWT has been performed on the wound site, etc.) is typically cumbersome, expensive, and inefficient due to the inability of the port to facilitate access to the wound site without being removed.

### Set Up of a NPWT System Using an Example Releasable Connector

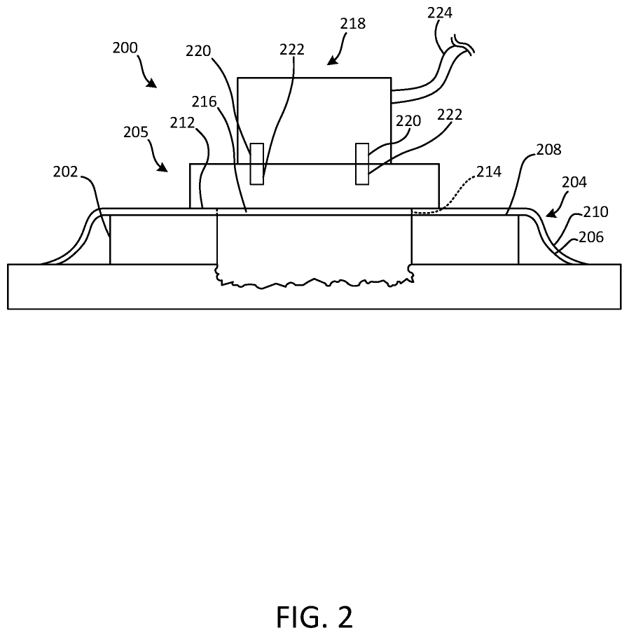

Referring now to FIG. 2, a releasable connector 200 for a NPWT system is shown, according to an exemplary embodiment. The NPWT system may be set up (e.g., installed, initially configured, etc.) to begin NPWT on a tissue wound. To begin set up of the NPWT system, a dressing 202 is first applied around and/or over the tissue wound.

Next, a drape 204 is placed over dressing 202. Drape 204 includes a bottom surface 206 which interfaces with a top surface 208 of dressing 202. Drape 204 also includes a top surface 210 opposite bottom surface 206. While not shown in FIG. 2, it is understood that in some embodiments, a cutting template may be placed on top of dressing 202 prior to drape 204 being placed over dressing 202. Drape 204 may then be cut around the cutting template to form an aperture through which the cutting template and a cut-out of the drape are subsequently removed.

After drape 204 has been placed over dressing 202, a connector base 205 of the releasable connector 200 is installed. The connector base 205 includes a bottom surface 212 that is configured to be coupled (e.g., affixed, attached, adhered, bonded, etc.) to top surface 210 such that a substantially air-tight seal exists between bottom surface 212 and top surface 210. In some embodiments, bottom surface 212 includes adhesive (e.g., glue, bonding agent, etc.) that is configured to facilitate coupling of bottom surface 212 to top surface 210.

Next, after bottom surface 212 is coupled to drape 204, an aperture 214 (e.g., opening, hole, etc.) is cut into drape 204. For example, a scalpel (e.g., knife, blade, instrument, etc.) may be inserted through an aperture within connector base 205, such that the scalpel contacts drape 204, and may be subsequently drawn against the aperture within the connector base 205, thereby forming aperture 214. After aperture 214 is formed, the scalpel may be removed from connector base 205 and a cut-out 216 of drape 204 may be removed through the aperture in connector base 205 (e.g., using a forceps, etc.), thereby forming aperture 214.

After aperture 214 has been formed, a fitting 218 (e.g., tube fitting, lens fitting, instillation connection fitting, etc.) is coupled to connector base 205 such that a substantially air-tight seal is formed between fitting 218 and connector base 205. Connector base 205 includes a connector coupling member 220 (e.g., projection, receptacle, etc.) and fitting 218 includes a fitting coupling member 222 (e.g., receptacle, projection, etc.). Connector coupling member 220 and fitting coupling member 222 are configured to cooperate couple connector base 205 and fitting 218. In various embodiments, connector coupling member 220 is a projection and fitting coupling member 222 is a receptacle. In other embodiments, connector coupling member 220 is a receptacle and fitting coupling member 222 is a projection. In some applications, the wound site is interacted with prior to fitting 218 being inserted into, and received by, connector base 205.

In some applications (e.g., applications where the fitting 218 is a tube fitting, applications where the fitting 218 is an instillation connection fitting, etc.), tubing 224 is next coupled to fitting 218. Tubing 224 may be similar to tubing 106 previously described. Tubing 224 may be coupled to a therapy device similar to therapy device 102.

### First Example Releasable Connector

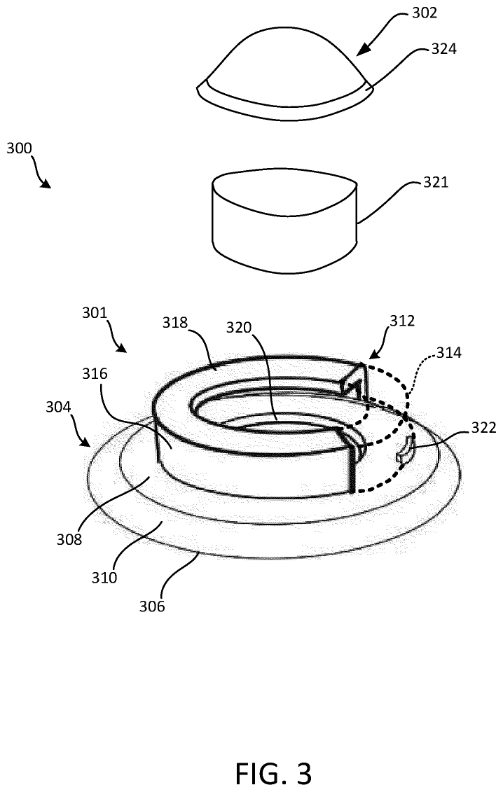

FIGS. 3-5 illustrate a releasable connector 300 having a connector base 301 and a fitting 302, according to an exemplary embodiment. Connector base 205 is connector base 301 and fitting 218 is fitting 302 in some embodiments.

Connector base 301 includes a base 304. Base 304 includes a bottom surface 306 that is configured to be coupled to top surface 210. In some embodiments, bottom surface 306 includes adhesive (e.g., glue, bonding agent, etc.) that is configured to facilitate coupling of bottom surface 306 to top surface 210. Base 304 also includes a top surface 308 opposite bottom surface 306 and a beveled surface 310 contiguous with (e.g., sharing a border with, etc.) both top surface 308 and bottom surface 306.

Base 304 also includes a base locking member 312. Base locking member 312 is configured to receive fitting 302 such that a substantially air-tight seal is formed between fitting 302 and top surface 308, thereby forming a substantially air-tight seal between drape 204 and fitting 302. Base locking member 312 defines a receiving opening 314. Base locking member 312 receives fitting 302 via an insertion of fitting 302 through receiving opening 314.

Base locking member 312 includes a projection 316 (e.g., protuberance, extension, etc.). Projection 316 extends (e.g., protrudes, projects, etc.) from top surface 308. Base locking member 312 also includes a flange 318 (e.g., extension, etc.). Flange 318 is contiguous with, and extends from, projection 316. Base locking member 312 also includes an aperture 320 (e.g., opening, hole, etc.). Aperture 320 is at least partially surrounded by (e.g., bordered by, encircled, etc.) projection 316 and receiving opening 314. Aperture 320 facilitates fluid communication through connector base 301. For example, when bottom surface 306 is coupled to drape 204 and fitting 302 is coupled to connector base 301 via base locking member 312, aperture 320 may facilitate fluid communication between tubing, such as tubing 224, coupled to fitting 302 and a wound site underneath drape 204.

In some embodiments, a removable manifold core 321 may be inserted through aperture 320 and maintained between fitting 302 and the wound bed. Removable manifold core 321 may absorb wound exudate and/or may be configured to deliver a treatment to the wound bed. After sufficient wound exudate has been absorbed by removable manifold core 321, or after sufficient treatment has been delivered by removable manifold core 321 to the wound bed, fitting 302 may be removed from connector base 301, removable manifold core 321 may be removed via aperture 320, and fitting 302 may be inserted into connector base 301.

Base locking member 312 also includes a locking member 322 (e.g., lug, lock, projection, protuberance, bump, etc.). Locking member 322 extends from top surface 308 and is configured to interface with fitting 302, when fitting 302 is received base locking member 312, to retain (e.g., secure, capture, etc.) fitting 302 relative to connector base 301.

Fitting 302 includes an interfacing flange 324. Interfacing flange 324 is configured to be received within base locking member 312. When interfacing flange 324 is received within base locking member 312, interfacing flange 324 is located between flange 318 and top surface 308. A substantially air-tight seal is formed between interfacing flange 324 and top surface 308 and a substantially air-tight seal is formed between interfacing flange 324 and flange 318 when interfacing flange 324 is received within base locking member 312. When interfacing flange 324 is received within base locking member 312, interfacing flange 324 may be biased against projection 316 by locking member 322.

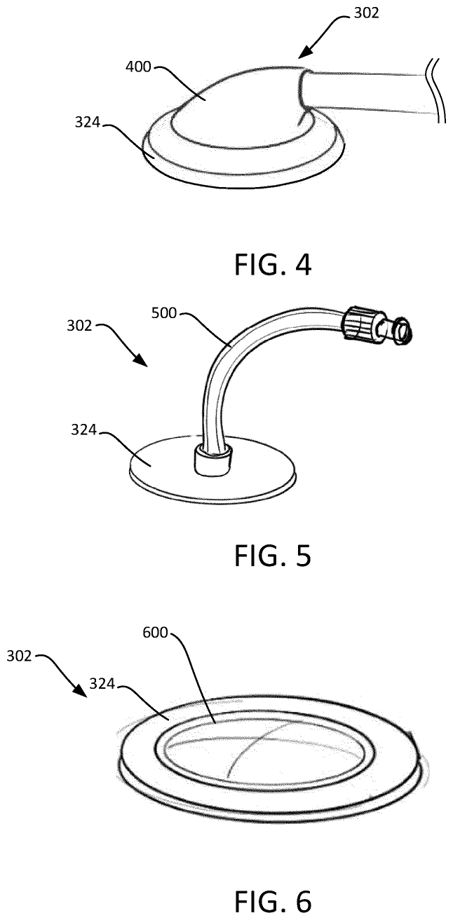

FIG. 4 illustrates fitting 302 as a tube connection fitting. In FIG. 4, fitting 302 also includes a tube connector 400. Tube connector 400 is configured to be coupled to tubing, such as tubing 224, and to facilitate fluid communication between, for example, a therapy device, and a wound site when fitting 302 is received within base locking member 312.

FIG. 5 illustrates fitting 302 as a manual instillation connection fitting. In FIG. 5, fitting 302 also includes a manual instillation tube connector 500. Manual instillation tube connector 500 is configured to be coupled to tubing, such as tubing 224, and to facilitate fluid communication between, for example, a manual instillation device (e.g., a hand pump, etc.), and a wound site when fitting 302 is received within base locking member 312.

FIG. 6 illustrates fitting 302 as a lens fitting. In FIG. 6, fitting 302 also includes a lens 600. Lens 600 is configured to facilitate viewing through fitting 302. For example, lens 600 may facilitate viewing of the wound bed underneath connector base 301. In some embodiments, lens 600 is configured to magnify (e.g., 1×, 3×, etc.) a view of the wound bed underneath connector base 301.

### Second Example Releasable Connector

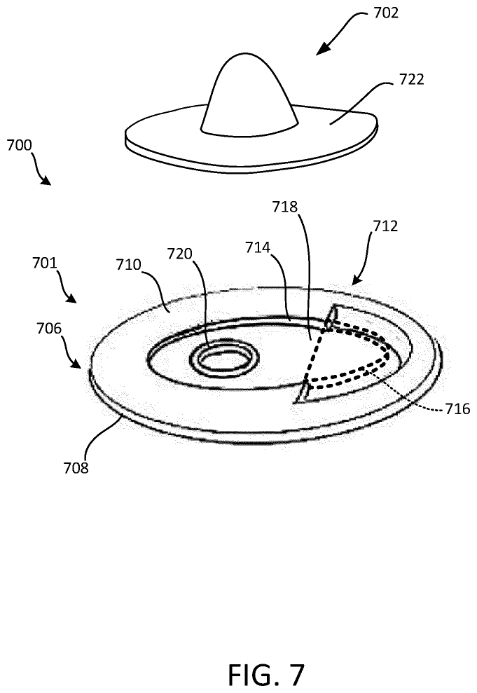

FIGS. 7-10 illustrate a releasable connector 700 having a connector base 701 and a fitting 702, according to an exemplary embodiment. Connector base 205 is connector base 701 and fitting 218 is fitting 702 in some embodiments.

Connector base 701 includes a base 706. Base 706 includes a bottom surface 708 that is configured to be coupled to top surface 210. In some embodiments, bottom surface 708 includes adhesive (e.g., glue, bonding agent, etc.) that is configured to facilitate coupling of bottom surface 708 to top surface 210.

Base 706 also includes a base locking member 712. Base locking member 712 is configured to receive fitting 702 such that a substantially air-tight seal is formed between fitting 702 and top surface 710, thereby forming a substantially air-tight seal between drape 204 and fitting 702.

Base locking member 712 includes a first flange 714, which defines a receiving opening 716, and a second flange 718. A portion of top surface 710 is a surface (e.g., a top surface, etc.) of first flange 714 and a portion of bottom surface 708 is a surface (e.g., a bottom surface, etc.) of second flange 718. In this way, connector base 701 may be substantially flat along both bottom surface 708 and top surface 710.

Second flange 718 is substantially parallel to first flange 714. Base locking member 712 receives fitting 702 via an insertion of fitting 702 through receiving opening 716 such that fitting 702 is captured between first flange 714 and second flange 718. When fitting 702 is received in base locking member 712, an interaction between first flange 714 and fitting 702 forms a substantially air-tight seal between first flange 714 and fitting 702 and an interaction between second flange 718 and fitting 702 forms a substantially air-tight seal between second flange 718 and fitting 702.

Base locking member 712 includes an aperture 720 (e.g., opening, hole, etc.). Aperture 720 is disposed on second flange 718. Aperture 720 facilitates fluid communication through connector base 701. For example, when bottom surface 708 is coupled to drape 204 and fitting 702 is coupled to connector base 701 via base locking member 712, aperture 720 may facilitate fluid communication between tubing, such as tubing 224, coupled to fitting 702 and a wound site underneath drape 204.

Fitting 702 includes an interfacing flange 722. Interfacing flange 722 is configured to be received within base locking member 712. When interfacing flange 722 is received within base locking member 712, interfacing flange 722 is located between first flange 714 and second flange 718. A substantially air-tight seal is formed between interfacing flange 722 and first flange 714 and a substantially air-tight seal is formed between interfacing flange 722 and second flange 718 when interfacing flange 722 is received within base locking member 712.

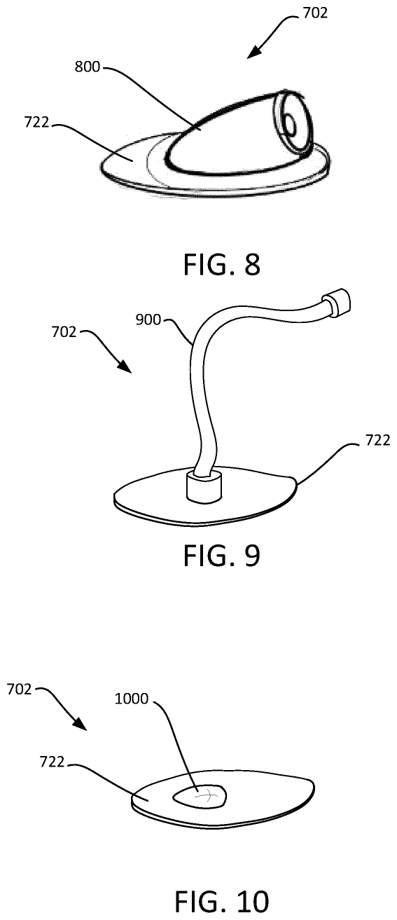

FIG. 8 illustrates fitting 702 as a tube connection fitting. In FIG. 8, fitting 702 also includes a tube connector 800. Tube connector 800 is configured to be coupled to tubing, such as tubing 224, and to facilitate fluid communication between, for example, a therapy device, and a wound site when fitting 702 is received within base locking member 712.

FIG. 9 illustrates fitting 702 as a manual instillation connection fitting. In FIG. 9, fitting 702 also includes a manual instillation tube connector 900. Manual instillation tube connector 900 is configured to be coupled to tubing, such as tubing 224, and to facilitate fluid communication between, for example, a manual instillation device (e.g., a hand pump, etc.), and a wound site when fitting 702 is received within base locking member 712.

FIG. 10 illustrates fitting 702 as a lens fitting. In FIG. 10, fitting 702 also includes a lens 1000. Lens 1000 is configured to facilitate viewing through fitting 702. For example, lens 1000 may facilitate viewing of the wound bed underneath connector base 701. In some embodiments, lens 1000 is configured to magnify (e.g., 1×, 3×, etc.) a view of the wound bed underneath connector base 701.

### Third Example Releasable Connector

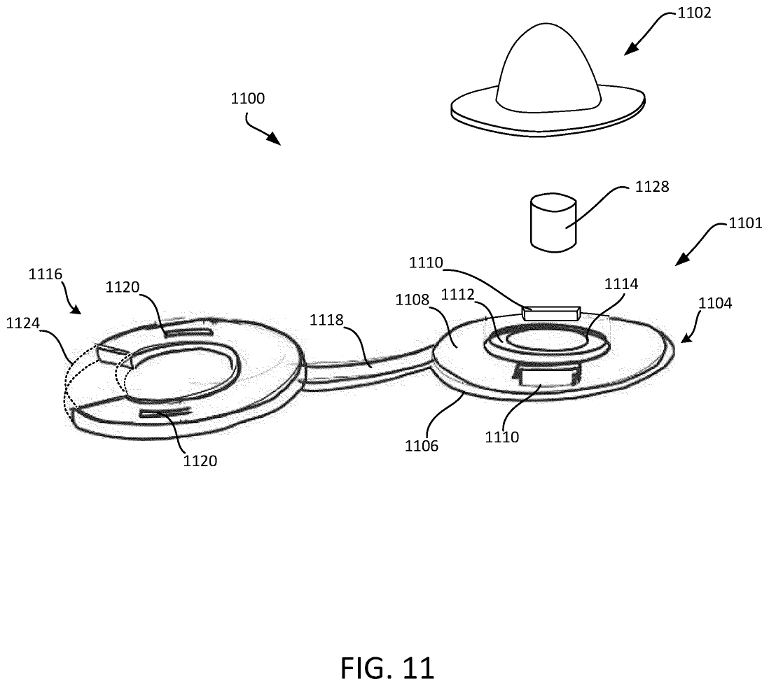

FIGS. 11-14 illustrate a releasable connector 1100 having a connector base 1101 and a fitting 1102, according to an exemplary embodiment. Connector base 205 is connector base 1101 and fitting 218 is fitting 1102 in some embodiments.

Connector base 1101 includes a base 1104. Base 1104 includes a bottom surface 1106 that is configured to be coupled to top surface 210. In some embodiments, bottom surface 1106 includes adhesive (e.g., glue, bonding agent, etc.) that is configured to facilitate coupling of bottom surface 1106 to top surface 210. Base 1104 also includes a top surface 1108 opposite bottom surface 1106.

Base 1104 includes locking protrusions 1110 (e.g., projections, extensions, protuberances, etc.) disposed on and extending from top surface 1108. Base 1104 also includes a center protrusion 1112 (e.g., projection, extension, protuberance, etc.) disposed on and extending from top surface 1108.

Base 1104 also includes an aperture 1114 (e.g., opening, hole, etc.). Aperture 1114 is disposed within center protrusion 1112. Aperture 1114 facilitates fluid communication through connector base 1101. For example, when bottom surface 1106 is coupled to drape 204 and fitting 1102 is coupled to connector base 1101, aperture 1114 may facilitate fluid communication between tubing, such as tubing 224, coupled to fitting 1102 and a wound site underneath drape 204.

Connector base 1101 also includes a base locking member 1116. Base locking member 1116 is coupled to base 1104 via a connecting member 1118 (e.g., strap, band, cable, wire, band, etc.). Connecting member 1118 may be structurally integrated with, or coupled to, base 1104 and base locking member 1116. Base locking member 1116 and base 1104 are configured to be selectively coupled together. When base locking member 1116 is coupled to base 1104, fitting 1102 may be captured therebetween such that a substantially air-tight seal is formed between fitting 1102 and base 1104 and a substantially air-tight seal is formed between base locking member 1116 and fitting 1102.

Base locking member 1116 includes locking receivers 1120 (e.g., indentations, slots, etc.). Locking receivers 1120 are configured to be aligned with, receive, and be coupled to locking protrusions 1110. When base locking member 1116 is brought into contact with base 1104, locking receivers 1120 are aligned with locking protrusions 1110 and locking protrusions 1110 are subsequently inserted into locking receivers 1120. In some embodiments, insertion of locking protrusions 1110 into locking receivers 1120 causes base locking member 1116 to be coupled to base 1104. In other embodiments, base locking member 1116 is coupled to base 1104 through rotation of base locking member 1116 relative to base 1104 once locking protrusions 1110 are inserted into locking receivers 1120.

Base locking member 1116 also includes an aperture 1122 (e.g., opening, hole, etc.). When fitting 1102 is captured between base 1104 and base locking member 1116, a portion of fitting 1102 protrudes through aperture 1122. Base locking member 1116 also defines a receiving opening 1124. Base locking member 1116 receives fitting 1102 via an insertion of fitting 1102 through receiving opening 1124. For example, fitting 1102 may be placed onto base 1104 and base locking member 1116 may subsequently be placed over fitting 1102, thereby sandwiching fitting 1102 between base 1104 and base locking member 1116.

In some embodiments, a removable manifold core 1128 may be inserted through aperture 1122 and maintained between fitting 1102 and the wound bed. Removable manifold core 1128 may absorb wound exudate and/or may be configured to deliver a treatment to the wound bed. After sufficient wound exudate has been absorbed by removable manifold core 1128, or after sufficient treatment has been delivered by removable manifold core 1128 to the wound bed, fitting 1102 may be removed from connector base 1101, removable manifold core 1128 may be removed via aperture 1122, and fitting 1102 may be inserted into connector base 1101.

Fitting 1102 includes an interfacing flange 1130. Interfacing flange 1130 is configured to be captured between base 1104 and base locking member 1116 when base locking member 1116 is coupled to base 1104. A substantially air-tight seal is formed between interfacing flange 1130 and base 1104 and a substantially air-tight seal is formed between interfacing flange 1130 and base locking member 1116 when fitting 1102 is captured between base 1104 and base locking member 1116.

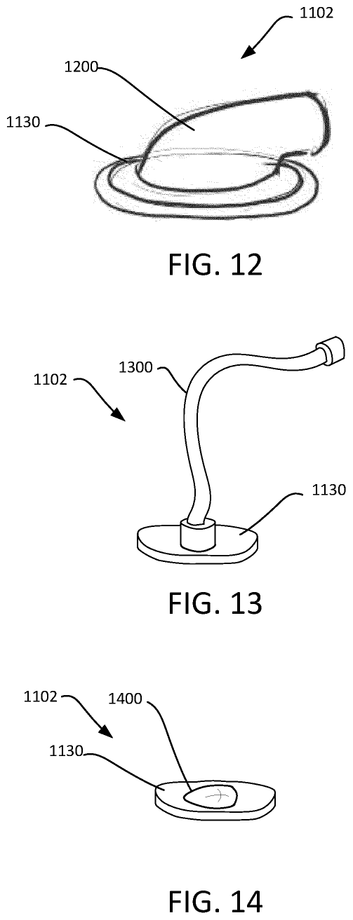

FIG. 12 illustrates fitting 1102 as a tube connection fitting. In FIG. 12, fitting 1102 also includes a tube connector 1200. Tube connector 1200 is configured to be coupled to tubing, such as tubing 224, and to facilitate fluid communication between, for example, a therapy device, and a wound site when fitting 1102 is captured between base 1104 and base locking member 1116.

FIG. 13 illustrates fitting 1102 as a manual instillation connection fitting. In FIG. 13, fitting 1102 also includes a manual instillation tube connector 1300. Manual instillation tube connector 1300 is configured to be coupled to tubing, such as tubing 224, and to facilitate fluid communication between, for example, a manual instillation device (e.g., a hand pump, etc.), and a wound site when fitting 1102 is received within base locking member 1116.

FIG. 14 illustrates fitting 1102 as a lens fitting. In FIG. 14, fitting 1102 also includes a lens 1400. Lens 1400 is configured to facilitate viewing through fitting 1102. For example, lens 1400 may facilitate viewing of the wound bed underneath connector base 1101. In some embodiments, lens 1400 is configured to magnify (e.g., 1×, 3×, etc.) a view of the wound bed underneath connector base 1101.

### Fourth Example Releasable Connector

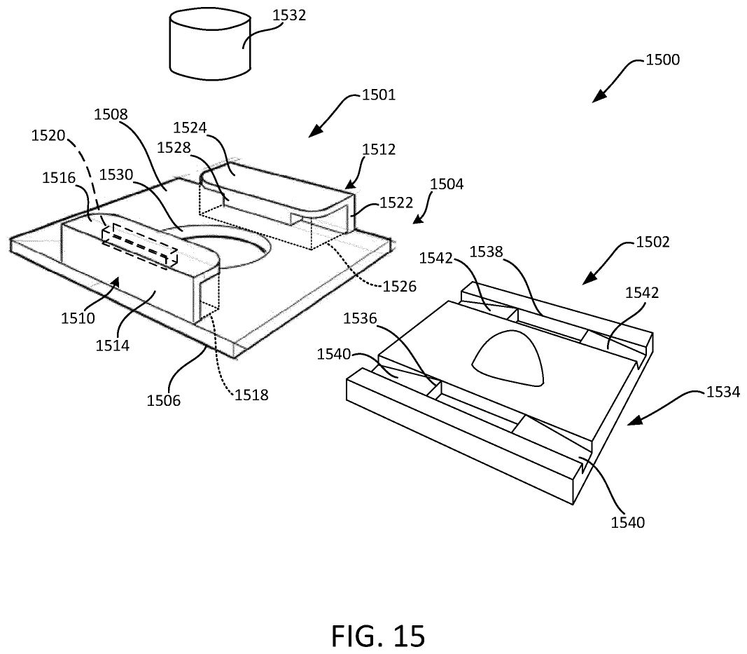

FIGS. 15-18 illustrate a releasable connector 1500 having a connector base 1501 and a fitting 1502, according to an exemplary embodiment. Connector base 205 is connector base 1501 and fitting 218 is fitting 1502 in some embodiments.

Connector base 1501 includes a base 1504. Base 1504 includes a bottom surface 1506 that is configured to be coupled to top surface 210. In some embodiments, bottom surface 1506 includes adhesive (e.g., glue, bonding agent, etc.) that is configured to facilitate coupling of bottom surface 1506 to top surface 210. Base 1504 includes a top surface 1508 opposite bottom surface 1506.

Base 1504 also includes a first base locking member 1510 and a second base locking member 1512. Second base locking member 1512 is identical to first base locking member 1510. First base locking member 1510 and second base locking member 1512 are configured to cooperate with base 1504 to receive fitting 1502 such that a substantially air-tight seal is formed between fitting 1502 and top surface 1508.

First base locking member 1510 includes a first projection 1514 and a first flange 1516 which collectively define a first receiving opening 1518. First base locking member 1510 also includes a first locking member 1520. First locking member 1520 extends from first flange 1516 towards top surface 1508.

Second base locking member 1512 includes a second projection 1522 and a second flange 1524 which collectively define a second receiving opening 1526. Second base locking member 1512 also includes a second locking member 1528. Second locking member 1528 extends from second flange 1524 towards top surface 1508.

First flange 1516 is substantially parallel to second flange 1524 and first projection 1514 is substantially parallel to second projection 1522. In various embodiments, first flange 1516 extends orthogonally from first projection 1514 and second flange 1524 extends orthogonally from second projection 1522.

First base locking member 1510 receives fitting 1502 in first receiving opening 1518 through an interaction between first locking member 1520 and fitting 1502 and second base locking member 1512 receives fitting 1502 in second receiving opening 1526 through an interaction between second locking member 1528 and fitting 1502. The interaction between first locking member 1520 and fitting 1502 and the interaction between second locking member 1528 and fitting 1502 couples fitting 1502 to base 1504 and forms a substantially air-tight seal between fitting 1502 and connector base 1501.

Base 1504 also includes an aperture 1530 (e.g., opening, hole, etc.). Aperture 1530 is disposed between first base locking member 1510 and second base locking member 1512. Aperture 1530 facilitates fluid communication through connector base 1501. For example, when bottom surface 1506 is coupled to drape 204 and fitting 1502 is coupled to connector base 1501 via first base locking member 1510 and second base locking member 1512, aperture 1530 may facilitate fluid communication between tubing, such as tubing 224, coupled to fitting 1502 and a wound site underneath drape 204.

In some embodiments, a removable manifold core 1532 may be inserted through aperture 1530 and maintained between fitting 1502 and the wound bed. Removable manifold core 1532 may absorb wound exudate and/or may be configured to deliver a treatment to the wound bed. After sufficient wound exudate has been absorbed by removable manifold core 1532, or after sufficient treatment has been delivered by removable manifold core 1532 to the wound bed, fitting 1502 may be removed from connector base 1501, removable manifold core 1532 may be removed via aperture 1530, and fitting 1302 may be inserted into connector base 1501.

Fitting 1502 includes an interfacing flange 1534. Interfacing flange 1534 is configured to be received within first receiving opening 1518 and second receiving opening 1526. Interfacing flange 1534 includes a first locking member receiver 1536 and a second locking member receiver 1538. First locking member receiver 1536 includes ramped portions 1540 and second locking member receiver 1538 includes ramped portions 1542. First locking member receiver 1536 is configured to receive first locking member 1520 and second locking member receiver 1538 is configured to receive second locking member 1528 such that when fitting 1502 is coupled to base 1504, first locking member 1520 is received (e.g., positioned, captured, etc.) within first locking member receiver 1536 and second locking member 1528 is received within second locking member receiver 1538. Ramped portions 1540 are configured to interface with first locking member 1520 to guide first locking member 1520 into first locking member receiver 1536. Ramped portions 1542 are configured to interface with second locking member 1528 to guide second locking member 1528 into second locking member receiver 1538.

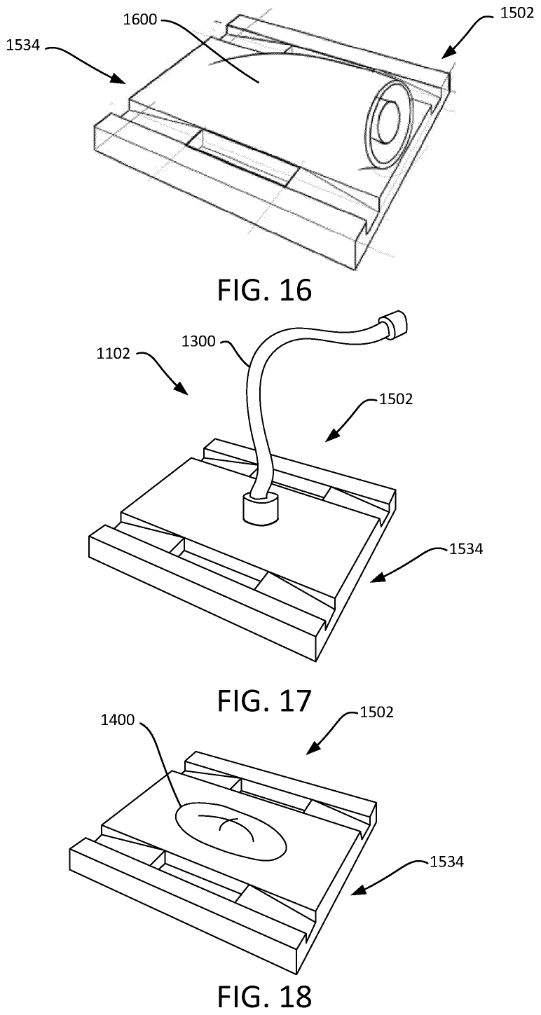

FIG. 16 illustrates fitting 1502 as a tube connection fitting. In FIG. 15, fitting 1502 also includes a tube connector 1600. Tube connector 1600 is configured to be coupled to tubing, such as tubing 224, and to facilitate fluid communication between, for example, a therapy device, and a wound site when fitting 1502 is coupled to connector base 1501.

FIG. 17 illustrates fitting 1502 as a manual instillation connection fitting. In FIG. 17, fitting 1502 also includes a manual instillation tube connector 1700. Manual instillation tube connector 1700 is configured to be coupled to tubing, such as tubing 224, and to facilitate fluid communication between, for example, a manual instillation device (e.g., a hand pump, etc.), and a wound site when fitting 1502 is coupled to connector base 1501.

FIG. 18 illustrates fitting 1502 as a lens fitting. In FIG. 18, fitting 1502 also includes a lens 1800. Lens 1800 is configured to facilitate viewing through fitting 1502. For example, lens 1800 may facilitate viewing of the wound bed underneath connector base 1501.

In some embodiments, lens 1800 is configured to magnify (e.g., 1×, 3×, etc.) a view of the wound bed underneath connector base 1501.

### Fifth Example Releasable Connector

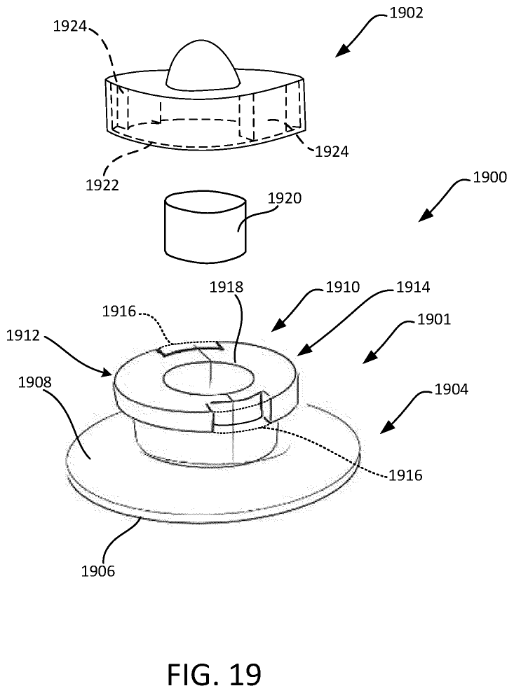

FIGS. 19-22 illustrate a releasable connector 1900 having a connector base 1901 and a fitting 1902, according to an exemplary embodiment. Connector base 205 is connector base 1901 and fitting 218 is fitting 1902 in some embodiments.

Connector base 1901 includes a base 1904. Base 1904 includes a bottom surface 1906 that is configured to be coupled to top surface 210. In some embodiments, bottom surface 1906 includes adhesive (e.g., glue, bonding agent, etc.) that is configured to facilitate coupling of bottom surface 1906 to top surface 210. Base 1904 includes a top surface 1908 opposite bottom surface 1906.

Base 1904 also includes a center protrusion 1910 extending from top surface 1908. Center protrusion 1910 includes a first base locking member 1912 and a second base locking member 1914. First base locking member 1912 and second base locking member 1914 are configured to be received within fitting 1902 such that, when first base locking member 1912 and second base locking member 1914 are received within fitting 1902, a substantially water-tight seal is formed between connector base 1901 and fitting 1902. In various embodiments, first base locking member 1912 is identical to second base locking member 1914. In some embodiments, first base locking member 1912 and second base locking member 1914 are disposed along at least one plane that is substantially parallel to top surface 1908.

First base locking member 1912 and second base locking member 1914 are configured such that openings 1916 (e.g., slots, apertures, windows, etc.) are interspaced between first base locking member 1912 and second base locking member 1914. Openings 1916 receive a portion of fitting 1902 such that center protrusion 1910 can be received within fitting 1902. Once center protrusion 1910 is received within fitting 1902, fitting 1902 may be rotated relative to connector base 1901 such that first base locking member 1912 and second base locking member 1914 rotate within fitting 1902. Rotation (e.g., a quarter turn, a half turn, a full turn, etc.) of first base locking member 1912 and second base locking member 1914 within fitting 1902 may cause fitting 1902 to be coupled to connector base 1901 and form a substantially air-tight seal between fitting 1902 and connector base 1901.

Base 1904 also includes an aperture 1918 (e.g., opening, hole, etc.). Aperture 1918 is disposed within center protrusion 1910. For example, aperture 1918 may be centered on (e.g., concentric with, etc.) center protrusion 1910. Aperture 1918 facilitates fluid communication through connector base 1901. For example, when bottom surface 1906 is coupled to drape 204 and fitting 1902 is coupled to connector base 1901 via first base locking member 1912 and second base locking member 1914, aperture 1918 may facilitate fluid communication between tubing, such as tubing 224, coupled to fitting 1902 and a wound site underneath drape 204.

In some embodiments, a removable manifold core 1920 may be inserted through aperture 1918 and maintained between fitting 1902 and the wound bed. Removable manifold core 1920 may absorb wound exudate and/or may be configured to deliver a treatment to the wound bed. After sufficient wound exudate has been absorbed by removable manifold core 1920, or after sufficient treatment has been delivered by removable manifold core 1920 to the wound bed, fitting 1902 may be removed from connector base 1901, removable manifold core 1920 may be removed via aperture 1918, and fitting 1902 may be inserted into connector base 1901.

Fitting 1902 includes an internal cavity 1922 that is configured to receive first base locking member 1912, second base locking member 1914, and center protrusion 1910. Fitting 1902 also includes locking projections 1924. Locking projections 1924 protrude (e.g., project, extend, etc.) into internal cavity 1922 and are configured to be aligned with and received within openings 1916. The number of locking projections 1924 is equal to the number of openings 1916.

When fitting 1902 is placed over connector base 1901, internal cavity 1922 may be substantially centered on center protrusion 1910. Fitting 1902 may then be pressed onto connector base 1901 and rotated such that locking projections 1924 align with openings 1916. After locking projections 1924 and openings 1916 are aligned, fitting 1902 may be further pressed onto connector base 1901 and subsequently rotated such that locking projections 1924 are not aligned with openings 1916 and are instead are aligned with first base locking member 1912 and second base locking member 1914. An interaction between first base locking member 1912 and associated locking projections 1924 and an interaction between second base locking member 1914 and associated locking projections 1924 causes fitting 1902 to be coupled to connector base 1901 such that a substantially air-tight seal is formed between fitting 1902 and connector base 1901.

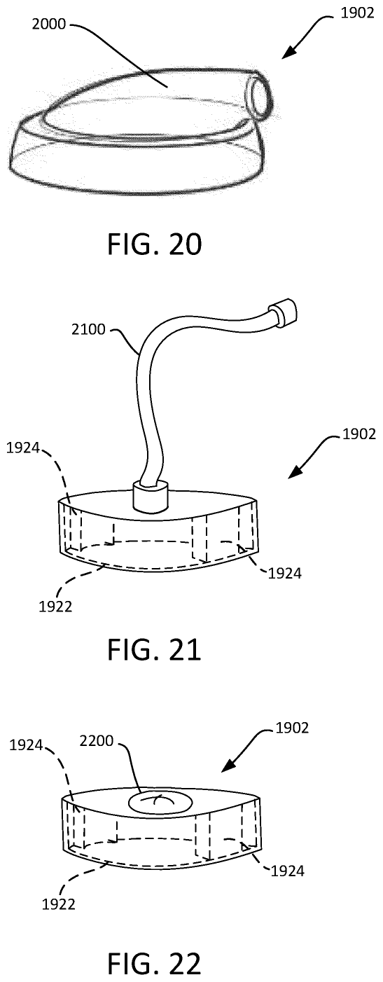

FIG. 20 illustrates fitting 1902 as a tube connection fitting. In FIG. 20, fitting 1902 also includes a tube connector 2000. Tube connector 2000 is configured to be coupled to tubing, such as tubing 224, and to facilitate fluid communication between, for example, a therapy device, and a wound site when fitting 1902 is coupled to connector base 1901.

FIG. 21 illustrates fitting 1902 as a manual instillation connection fitting. In FIG. 21, fitting 1902 also includes a manual instillation tube connector 2100. Manual instillation tube connector 2100 is configured to be coupled to tubing, such as tubing 224, and to facilitate fluid communication between, for example, a manual instillation device (e.g., a hand pump, etc.), and a wound site when fitting 1902 is coupled to connector base 1901.

FIG. 22 illustrates fitting 1902 as a lens fitting. In FIG. 22, fitting 1902 also includes a lens 2200. Lens 2200 is configured to facilitate viewing through fitting 1902. For example, lens 2200 may facilitate viewing of the wound bed underneath connector base 1901. In some embodiments, lens 2200 is configured to magnify (e.g., 1×, 3×, etc.) a view of the wound bed underneath connector base 1901.

### Sixth Example Releasable Connector

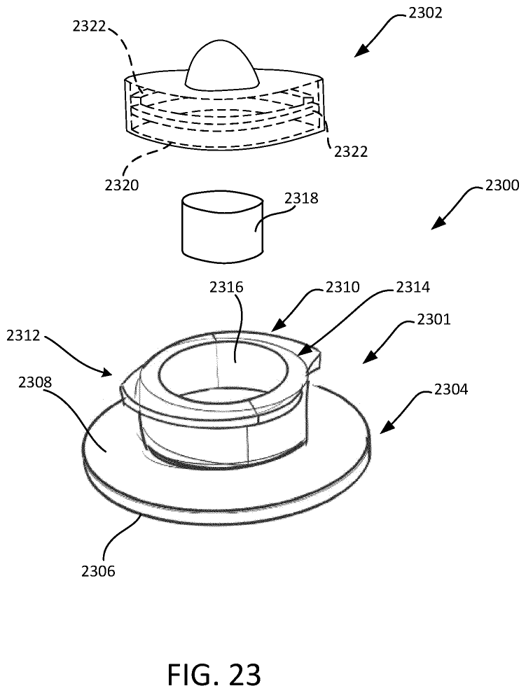

FIGS. 23-26 illustrate a releasable connector 2300 having a connector base 2301 and a fitting 2302, according to an exemplary embodiment. Connector base 205 is connector base 2301 and fitting 218 is fitting 2302 in some embodiments.

Connector base 2301 includes a base 2304. Base 2304 includes a bottom surface 2306 that is configured to be coupled to top surface 210. In some embodiments, bottom surface 2306 includes adhesive (e.g., glue, bonding agent, etc.) that is configured to facilitate coupling of bottom surface 2306 to top surface 210. Base 2304 includes a top surface 2308 opposite bottom surface 2306.

Base 2304 also includes a center protrusion 2310 extending from top surface 2308. Center protrusion 2310 includes a first base locking member 2312 and a second base locking member 2314. First base locking member 2312 and second base locking member 2314 are configured to be received within fitting 2302 such that, when first base locking member 2312 and second base locking member 2314 are received within fitting 2302, a substantially water-tight seal is formed between connector base 2301 and fitting 2302. In various embodiments, first base locking member 2312 is identical to second base locking member 2314. In some embodiments, first base locking member 2312 and second base locking member 2314 are disposed along at least one plane that is substantially parallel to top surface 2308.

Once center protrusion 2310 is received within fitting 2302, fitting 2302 may be rotated relative to connector base 2301 such that first base locking member 2312 and second base locking member 2314 rotate within fitting 2302. Rotation (e.g., a quarter turn, a half turn, a full turn, etc.) of first base locking member 2312 and second base locking member 2314 within fitting 2302 may cause fitting 2302 to be coupled to connector base 2301 and form a substantially air-tight seal between fitting 2302 and connector base 2301.

Base 2304 also includes an aperture 2316 (e.g., opening, hole, etc.). Aperture 2316 is disposed within center protrusion 2310. For example, aperture 2316 may be centered on (e.g., concentric with, etc.) center protrusion 2310. Aperture 2316 facilitates fluid communication through connector base 2301. For example, when bottom surface 2306 is coupled to drape 204 and fitting 2302 is coupled to connector base 2301 via first base locking member 2312 and second base locking member 2314, aperture 2316 may facilitate fluid communication between tubing, such as tubing 224, coupled to fitting 2302 and a wound site underneath drape 204.

In some embodiments, a removable manifold core 2318 may be inserted through aperture 2316 and maintained between fitting 2302 and the wound bed. Removable manifold core 2318 may absorb wound exudate and/or may be configured to deliver a treatment to the wound bed. After sufficient wound exudate has been absorbed by removable manifold core 2318, or after sufficient treatment has been delivered by removable manifold core 2318 to the wound bed, fitting 2302 may be removed from connector base 2301, removable manifold core 2318 may be removed via aperture 2316, and fitting 2302 may be inserted into connector base 2301.

Fitting 2302 includes an internal cavity 2320 that is configured to receive first base locking member 2312, second base locking member 2314, and center protrusion 2310. Fitting 2302 also includes locking projections 2322. Locking projections 2322 protrude (e.g., project, extend, etc.) into internal cavity 2320.

First base locking member 2312, second base locking member 2314, and locking projections 2322 are generally ramp shaped so that when locking projections 2322 are rotated against first base locking member 2312 and second base locking member 2314, locking projections 2322 interface with first base locking member 2312 and interface with second base locking member 2314 along an elongated area (e.g., the ramped face of first base locking member 2312 and the ramped face of second base locking member 2314, etc.).

Locking projections 2322 may include two parallel flanges forming a cavity therebetween. This cavity receives first base locking member 2312 and second base locking member 2314. The parallel flanges may be shaped to facilitate alignment of the first base locking member 2312 and second base locking member 2314 with the cavity.

When fitting 2302 is placed over connector base 2301, internal cavity 2320 may be substantially centered on center protrusion 2310. Fitting 2302 may then be pressed onto connector base 2301 and rotated such that locking projections 2322 are disposed between first base locking member 2312 and second base locking member 2314. Fitting 2302 may then be further pressed onto connector base 2301 and subsequently rotated such that locking projections 2322 contact first base locking member 2312 and second base locking member 2314. An interaction between first base locking member 2312 and associated locking projections 2322 and an interaction between second base locking member 2314 and associated locking projections 2322 causes fitting 2302 to be coupled to connector base 2301 such that a substantially air-tight seal is formed between fitting 2302 and connector base 2301.

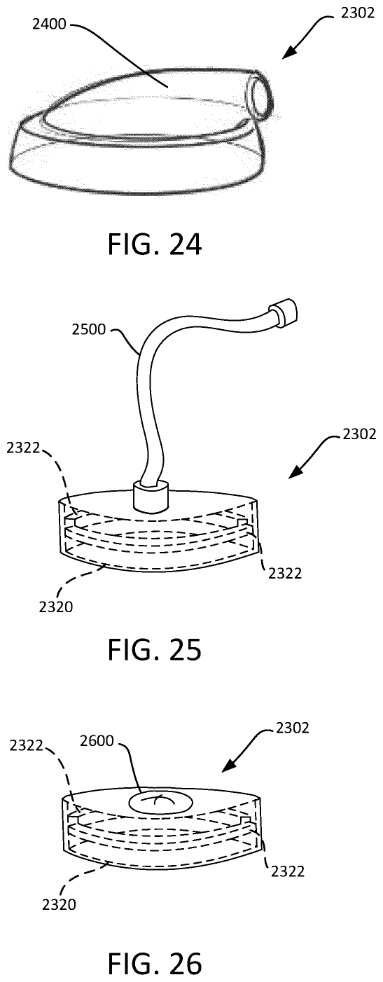

FIG. 24 illustrates fitting 2302 as a tube connection fitting. In FIG. 24, fitting 2302 also includes a tube connector 2400. Tube connector 2400 is configured to be coupled to tubing, such as tubing 224, and to facilitate fluid communication between, for example, a therapy device, and a wound site when fitting 2302 is coupled to connector base 2301.

FIG. 25 illustrates fitting 2302 as a manual instillation connection fitting. In FIG. 25, fitting 2302 also includes a manual instillation tube connector 2500. Manual instillation tube connector 2500 is configured to be coupled to tubing, such as tubing 224, and to facilitate fluid communication between, for example, a manual instillation device (e.g., a hand pump, etc.), and a wound site when fitting 2302 is coupled to connector base 2301.

FIG. 26 illustrates fitting 2302 as a lens fitting. In FIG. 26, fitting 2302 also includes a lens 2600. Lens 2600 is configured to facilitate viewing through fitting 2302. For example, lens 2600 may facilitate viewing of the wound bed underneath connector base 2301. In some embodiments, lens 2600 is configured to magnify (e.g., 1×, 3×, etc.) a view of the wound bed underneath connector base 2301.

### Seventh Example Releasable Connector

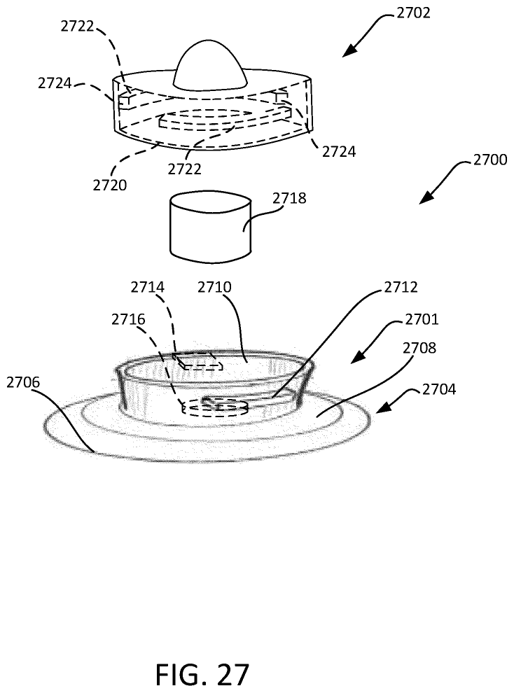

FIGS. 27-30 illustrate a releasable connector 2700 having a connector base 2701 and a fitting 2702, according to an exemplary embodiment. Connector base 205 is connector base 2701 and fitting 218 is fitting 2702 in some embodiments.

Connector base 2701 includes a base 2704. Base 2704 includes a bottom surface 2706 that is configured to be coupled to top surface 210. In some embodiments, bottom surface 2706 includes adhesive (e.g., glue, bonding agent, etc.) that is configured to facilitate coupling of bottom surface 2706 to top surface 210. Base 2704 includes a top surface 2708 opposite bottom surface 2706.

Base 2704 also includes a center protrusion 2710 extending from top surface 2708. Center protrusion 2710 includes a first base locking member 2712 and a second base locking member 2714. First base locking member 2712 and second base locking member 2714 are configured to be received within fitting 2702 such that, when first base locking member 2712 and second base locking member 2714 are received within fitting 2702, a substantially water-tight seal is formed between connector base 2701 and fitting 2702. In various embodiments, first base locking member 2712 is identical to second base locking member 2714. In some embodiments, first base locking member 2712 and second base locking member 2714 are disposed along at least one plane that is substantially parallel to top surface 2708.

Base 2704 also includes an aperture 2716 (e.g., opening, hole, etc.). Aperture 2716 is disposed within center protrusion 2710. For example, aperture 2716 may be centered on (e.g., concentric with, etc.) center protrusion 2710. Aperture 2716 facilitates fluid communication through connector base 2701. For example, when bottom surface 2706 is coupled to drape 204 and fitting 2702 is coupled to connector base 2701 via first base locking member 2712 and second base locking member 2714, aperture 2716 may facilitate fluid communication between tubing, such as tubing 224, coupled to fitting 2702 and a wound site underneath drape 204.

In some embodiments, a removable manifold core 2718 may be inserted through aperture 2716 and maintained between fitting 2702 and the wound bed. Removable manifold core 2718 may absorb wound exudate and/or may be configured to deliver a treatment to the wound bed. After sufficient wound exudate has been absorbed by removable manifold core 2718, or after sufficient treatment has been delivered by removable manifold core 2718 to the wound bed, fitting 2702 may be removed from connector base 2701, removable manifold core 2718 may be removed via aperture 2716, and fitting 2702 may be inserted into connector base 2701.

Fitting 2702 includes an internal cavity 2720 that is configured to receive first base locking member 2712, second base locking member 2714, and center protrusion 2710. Fitting 2702 also includes a flange 2722 having openings 2724 (e.g., slots, apertures, windows, etc.). Openings 2724 are configured to receive first base locking member 2712 and second base locking member 2714. Once center protrusion 2710 is received within fitting 2702, fitting 2702 may be rotated relative to connector base 2701 such that first base locking member 2712 and second base locking member 2714 rotate within fitting 2702. Rotation (e.g., a quarter turn, a half turn, a full turn, etc.) of first base locking member 2712 and second base locking member 2714 within fitting 2702 may cause fitting 2702 to be coupled to connector base 2701 and form a substantially air-tight seal between fitting 2702 and connector base 2701.

When fitting 2702 is placed over connector base 2701, internal cavity 2720 may be substantially centered on center protrusion 2710. Fitting 2702 may then be pressed onto connector base 2701 and rotated such that first base locking member 2712 and second base locking member 2714 are aligned with openings 2724. After first base locking member 2712 and second base locking member 2714 are aligned with openings 2724, fitting 2702 may be further pressed onto connector base 2701 and subsequently rotated such that first base locking member 2712 and second base locking member 2714 are not aligned with openings 2724 and are instead are aligned with flange 2722. An interaction between first base locking member 2712 and flange 2722 and an interaction between second base locking member 2714 and flange 2722 causes fitting 2702 to be coupled to connector base 2701 such that a substantially air-tight seal is formed between fitting 2702 and connector base 2701.

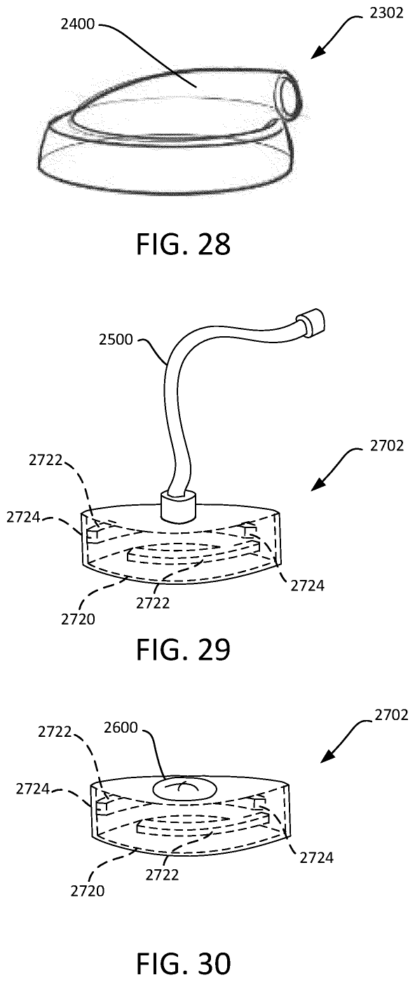

FIG. 28 illustrates fitting 2702 as a tube connection fitting. In FIG. 28, fitting 2702 also includes a tube connector 2800. Tube connector 2800 is configured to be coupled to tubing, such as tubing 224, and to facilitate fluid communication between, for example, a therapy device, and a wound site when fitting 2702 is coupled to connector base 2701.

FIG. 29 illustrates fitting 2702 as a manual instillation connection fitting. In FIG. 29, fitting 2702 also includes a manual instillation tube connector 2900. Manual instillation tube connector 2900 is configured to be coupled to tubing, such as tubing 224, and to facilitate fluid communication between, for example, a manual instillation device (e.g., a hand pump, etc.), and a wound site when fitting 2702 is coupled to connector base 2701.

FIG. 30 illustrates fitting 2702 as a lens fitting. In FIG. 30, fitting 2702 also includes a lens 3000. Lens 3000 is configured to facilitate viewing through fitting 2702. For example, lens 3000 may facilitate viewing of the wound bed underneath connector base 2701. In some embodiments, lens 3000 is configured to magnify (e.g., 1×, 3×, etc.) a view of the wound bed underneath connector base 2701.

### Eighth Example Releasable Connector

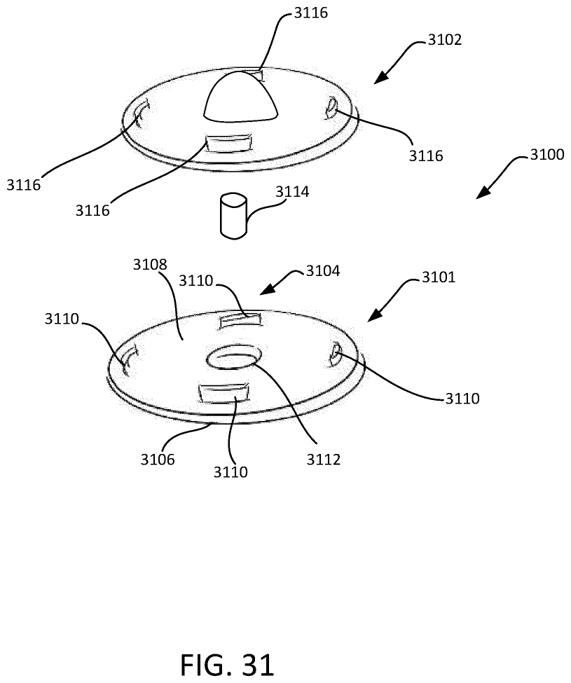

FIGS. 31-34 illustrate a releasable connector 3100 having a connector base 3101 and a fitting 3102, according to an exemplary embodiment. Connector base 205 is connector base 3101 and fitting 218 is fitting 3102 in some embodiments.

Connector base 3101 includes a base 3104. Base 3104 includes a bottom surface 3106 that is configured to be coupled to top surface 210. In some embodiments, bottom surface 3106 includes adhesive (e.g., glue, bonding agent, etc.) that is configured to facilitate coupling of bottom surface 3106 to top surface 210. Base 3104 includes a top surface 3108 opposite bottom surface 3106.

Base 3104 also includes a plurality of base locking members 3110 disposed on top surface 3108. In various embodiments, base locking members 3110 are snap locking members. Base locking members 3110 are configured to be received within fitting 3102 such that, when base locking members 3110 are received within fitting 3102, a substantially water-tight seal is formed between connector base 3101 and fitting 3102.

Base 3104 also includes an aperture 3112 (e.g., opening, hole, etc.). Aperture 3112 is disposed on top surface 3108. For example, aperture 3112 may be centered on (e.g., concentric with, etc.) top surface 3108. Aperture 3112 facilitates fluid communication through connector base 3101. For example, when bottom surface 3106 is coupled to drape 204 and fitting 3102 is coupled to connector base 3101 via base locking members 3110, aperture 3112 may facilitate fluid communication between tubing, such as tubing 224, coupled to fitting 3102 and a wound site underneath drape 204.

In some embodiments, a removable manifold core 3114 may be inserted through aperture 3112 and maintained between fitting 3102 and the wound bed. Removable manifold core 3114 may absorb wound exudate and/or may be configured to deliver a treatment to the wound bed. After sufficient wound exudate has been absorbed by removable manifold core 3114, or after sufficient treatment has been delivered by removable manifold core 3114 to the wound bed, fitting 3102 may be removed from connector base 3101, removable manifold core 3114 may be removed via aperture 3112, and fitting 3102 may be inserted into connector base 3101.

Fitting 3102 includes a plurality of fitting locking members 3116. Each of the fitting locking members 3116 is configured to receive and be coupled to one of the plurality of base locking members 3110. Depression (e.g., lowering, pressing, etc.) of fitting 3102 onto connector base 3101 causes each of fitting locking members 3116 to be coupled to one of base locking members 3110 and forms a substantially air-tight seal between fitting 3102 and connector base 3101.

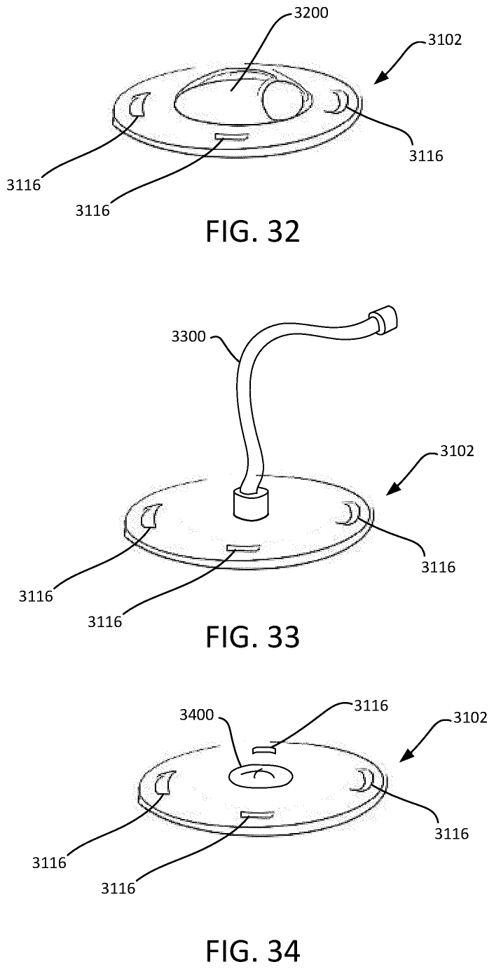

FIG. 32 illustrates fitting 3102 as a tube connection fitting. In FIG. 32, fitting 3102 also includes a tube connector 3200. Tube connector 3200 is configured to be coupled to tubing, such as tubing 224, and to facilitate fluid communication between, for example, a therapy device, and a wound site when fitting 3102 is coupled to connector base 3101.

FIG. 33 illustrates fitting 3102 as a manual instillation connection fitting. In FIG. 33, fitting 3102 also includes a manual instillation tube connector 3300. Manual instillation tube connector 3300 is configured to be coupled to tubing, such as tubing 224, and to facilitate fluid communication between, for example, a manual instillation device (e.g., a hand pump, etc.), and a wound site when fitting 3102 is coupled to connector base 3101.

FIG. 34 illustrates fitting 3102 as a lens fitting. In FIG. 34, fitting 3102 also includes a lens 3400. Lens 3400 is configured to facilitate viewing through fitting 3102. For example, lens 3400 may facilitate viewing of the wound bed underneath connector base 3101. In some embodiments, lens 3400 is configured to magnify (e.g., 1×, 3×, etc.) a view of the wound bed underneath connector base 3101.

### Ninth Example Releasable Connector

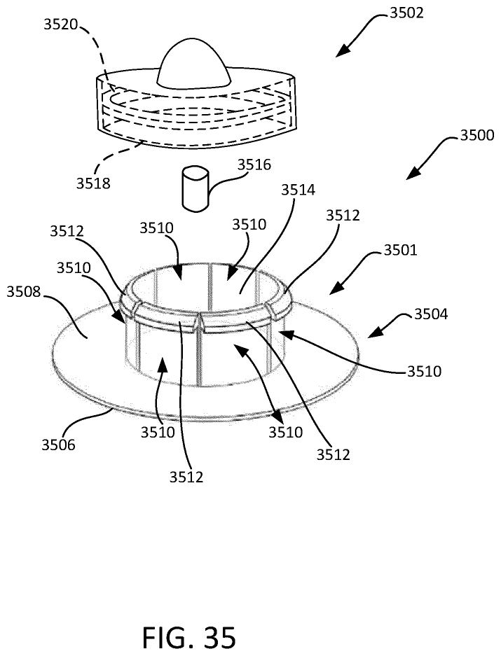

FIGS. 35-38 illustrate a releasable connector 3500 having a connector base 3501 and a fitting 3502, according to an exemplary embodiment. Connector base 205 is connector base 3501 and fitting 218 is fitting 3502 in some embodiments.

Connector base 3501 includes a base 3504. Base 3504 includes a bottom surface 3506 that is configured to be coupled to top surface 210. In some embodiments, bottom surface 3506 includes adhesive (e.g., glue, bonding agent, etc.) that is configured to facilitate coupling of bottom surface 3506 to top surface 210. Base 3504 includes a top surface 3508 opposite bottom surface 3506.

Base 3504 also includes a plurality of base locking members 3510 disposed on, and projecting from, top surface 3508. In various embodiments, base locking members 3510 are biasing members. Base locking members 3510 are configured to be received within fitting 3502 such that, when base locking members 3510 are received within fitting 3502, a substantially water-tight seal is formed between connector base 3501 and fitting 3502. Each base locking member 3510 includes a latch 3512 (e.g., catch, projection, etc.) that is configured to interface with fitting 3502. Latches 3512 project radially outward from each base locking member 3510. Additionally, each base locking member 3510 is configured to be displaced independent of the others of base locking member 3510.

Base 3504 also includes an aperture 3514 (e.g., opening, hole, etc.). Aperture 3514 is disposed on top surface 3508. For example, aperture 3514 may be centered on (e.g., concentric with, etc.) top surface 3508. Aperture 3514 facilitates fluid communication through connector base 3501. For example, when bottom surface 3506 is coupled to drape 204 and fitting 3502 is coupled to connector base 3501 via base locking members 3510, aperture 3514 may facilitate fluid communication between tubing, such as tubing 224, coupled to fitting 3502 and a wound site underneath drape 204. Aperture 3514 may be surrounded by (e.g., bordered by, contiguous with, etc.) base locking members 3510.

In some embodiments, a removable manifold core 3516 may be inserted through aperture 3514 and maintained between fitting 3502 and the wound bed. Removable manifold core 3516 may absorb wound exudate and/or may be configured to deliver a treatment to the wound bed. After sufficient wound exudate has been absorbed by removable manifold core 3516, or after sufficient treatment has been delivered by removable manifold core 3516 to the wound bed, fitting 3502 may be removed from connector base 3501, removable manifold core 3516 may be removed via aperture 3514, and fitting 3502 may be inserted into connector base 3501.

Fitting 3502 includes an internal cavity 3518 that is configured to receive base locking members 3510. Fitting 3502 also includes a locking flange 3520 that is configured to be coupled to base locking members 3510 via an interaction between latches 3512 and locking flange 3520. Locking flange 3520 may be an annular flange which extends about internal cavity 3518. Depression (e.g., lowering, pressing, etc.) of fitting 3502 onto connector base 3501 causes latches 3512 to interface with locking flange 3520. Interfacing of latches 3512 with locking flanges 3520 causes base locking members 3510 to be biased radially inwards until latches 3512 no longer are biased radially inwards by locking flange 3520, at which point base locking members 3510 deflect radially outwards. Each of latches 3512 is configured to be coupled to locking flange 3520 such that a substantially air-tight seal is formed between fitting 3502 and connector base 3501.

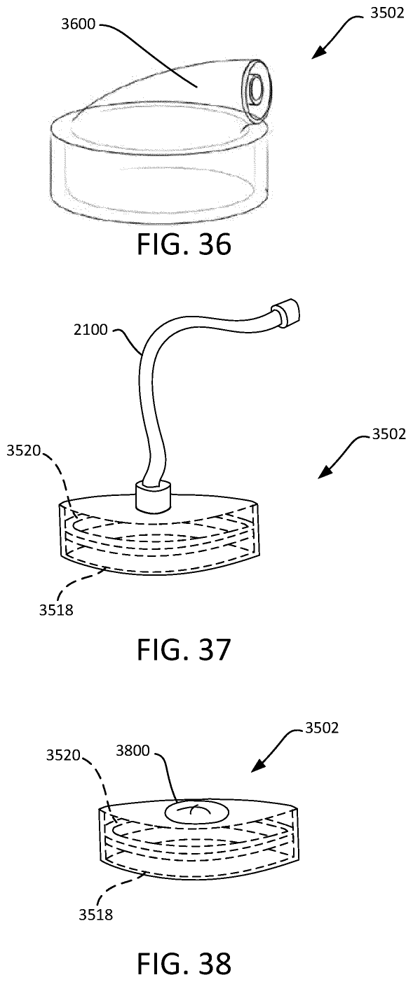

FIG. 36 illustrates fitting 3502 as a tube connection fitting. In FIG. 36, fitting 3502 also includes a tube connector 3600. Tube connector 3600 is configured to be coupled to tubing, such as tubing 224, and to facilitate fluid communication between, for example, a therapy device, and a wound site when fitting 3502 is coupled to connector base 3501.

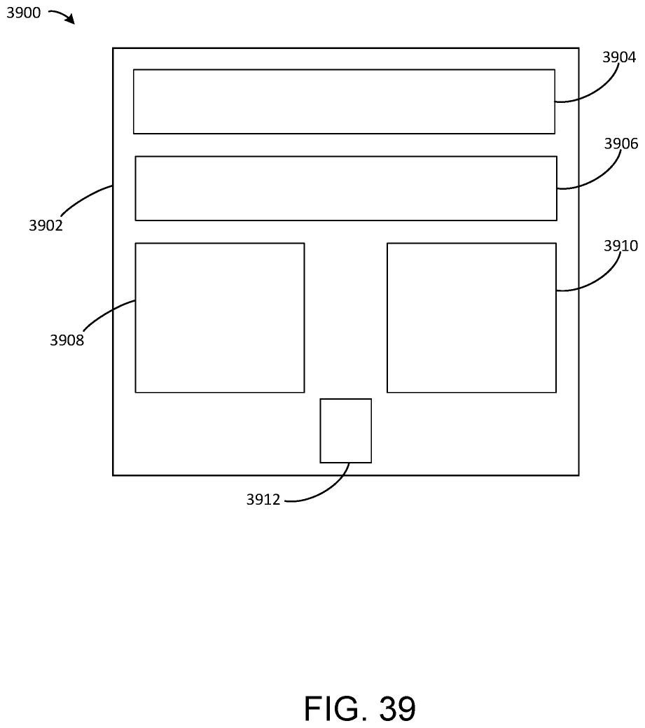

FIG. 37 illustrates fitting 3502 as a manual instillation connection fitting. In FIG. 36, fitting 3502 also includes a manual instillation tube connector 3700. Manual instillation tube connector 3700 is configured to be coupled to tubing, such as tubing 224, and to facilitate fluid communication between, for example, a manual instillation device (e.g., a hand pump, etc.), and a wound site when fitting 3502 is coupled to connector base 3501.

FIG. 38 illustrates fitting 3502 as a lens fitting. In FIG. 37, fitting 3502 also includes a lens 3800. Lens 3800 is configured to facilitate viewing through fitting 3502. For example, lens 3800 may facilitate viewing of the wound bed underneath connector base 3501. In some embodiments, lens 3800 is configured to magnify (e.g., 1×, 3×, etc.) a view of the wound bed underneath connector base 3501.

Kit for Use with a NPWT Device

Referring now to FIG. 39, a kit 3900 is shown, according to an exemplary embodiment. Kit 3900 is for use with a NPWT device, such as therapy device 102. Kit 3900 includes a package 3902. Package 3902 defines a sterilized interior environment. Package 3902 may be a bag, a case, and other similar structures. Kit 3900 also includes a dressing layer 3904. Dressing layer 3904 is disposed within package 3902. Dressing layer 3904 may be dressing 110 and dressing 202, alone or in combination. Kit 3900 also includes a drape layer 3906. Drape layer 3906 is disposed within package 3902. Drape layer 3906 may be drape 112 and drape 204, alone or in combination. Kit 3900 also includes a connector base 3908. Connector base 3908 is disposed within package 3902. Connector base 3908 may be any of connector base 205, connector base 301, connector base 701, connector base 1101, connector base 1501, connector base 1901, connector base 2301, connector base 2701, connector base 3101, and connector base 3501, alone or in combination. Kit 3900 also includes a fitting 3910. Fitting 3910 is disposed within package 3902. Fitting 3910 may be any of fitting 218, fitting 302, fitting 702, fitting 1102, fitting 1502, fitting 1902, fitting 2302, fitting 2702, fitting 3102, and fitting 3502, alone or in combination. Kit 3900 also includes a removable manifold core 3912. Removable manifold core 3912 is disposed within package 3902. Removable manifold core 3912 may be any of removable manifold core 321, removable manifold core 1128, removable manifold core 1532, removable manifold core 1920, removable manifold core 2318, removable manifold core 2718, removable manifold core 3114, and removable manifold core 3515, alone or in combination.

First Method of Using a NPWT Device with a Releasable Connector

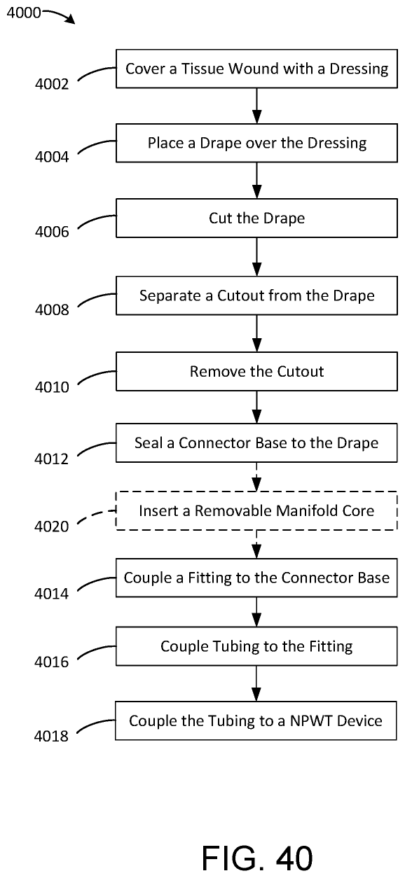

Referring now to FIG. 40, a method of using a NPWT device with a releasable connector 4000 is shown, according to an exemplary embodiment. Method 4000 is implemented with a NPWT device, such as therapy device 102. Method 4000 is implemented with a releasable connector and a fitting. The releasable connector includes a connector base and a fitting. The connector base may be any of connector base 205, connector base 301, connector base 701, connector base 1101, connector base 1501, connector base 1901, connector base 2301, connector base 2701, connector base 3101, and connector base 3501, alone or in combination. The fitting may be any of fitting 218, fitting 302, fitting 702, fitting 1102, fitting 1502, fitting 1902, fitting 2302, fitting 2702, fitting 3102, and fitting 3502, alone or in combination. Method 4000 may also be implemented with a removable manifold core. The removable manifold core may be any of removable manifold core 321, removable manifold core 1128, removable manifold core 1532, removable manifold core 1920, removable manifold core 2318, removable manifold core 2718, removable manifold core 3114, and removable manifold core 3515, alone or in combination.

Method 4000 includes, in block 4002, covering a tissue would with a dressing. The dressing may be placed over at least a portion of a wound bed or tissue wound. Method 4000 then includes, in block 4004, placing a drape over the dressing. The drape may only cover part of the dressing. Method 4000 then includes, in block 4006, cutting the drape. The drape may be cut by pressing an implement against the drape, thereby causing the drape to be cut. Method 4000 then includes, in block 4008, separating a cutout from the drape. Method 4000 then includes, in block 4010, removing the cutout from the drape. When the cutout is removed from the drape, an aperture remains in the drape. Method 4000 then includes, in block 4012, sealing (e.g., coupling, attaching, etc.) a connector base to the drape about the aperture such that a seal is formed between the drape and the connector base. For example, a removable backing may be removed from a bottom surface of the connector base, thereby exposing an adhesive disposed on the bottom surface. The connector base may then be pressed onto the drape such that the adhesive on the bottom surface bonds to the drape. Method 4000 then includes, in block 4014, coupling a fitting to the connector base such that a seal is formed between the fitting and the connector base. For example, the fitting may be coupled to the connector base via an interaction between an interfacing flange of the fitting and a base locking member of the connector base. Method 4000 then includes, in block 4016, coupling tubing to the fitting such that a seal is formed between the fitting and the tubing. For example, the tubing may be coupled to the fitting via a mating interaction between the fitting and tubing. Method 4000 finally includes, in block 4018, coupling the tubing to a NPWT device such that a seal is formed between the tubing and the NPWT device.

Method 4000 may also include, prior to coupling the fitting to the connector base and after sealing the connector base to the drape, in block 4020, inserting a removable manifold core into the connector base. When the fitting is coupled to the connector base, in block 4014, the removable manifold core may be captured between the fitting and the dressing or tissue wound.

Second Method of Using a NPWT Device with a Releasable Connector

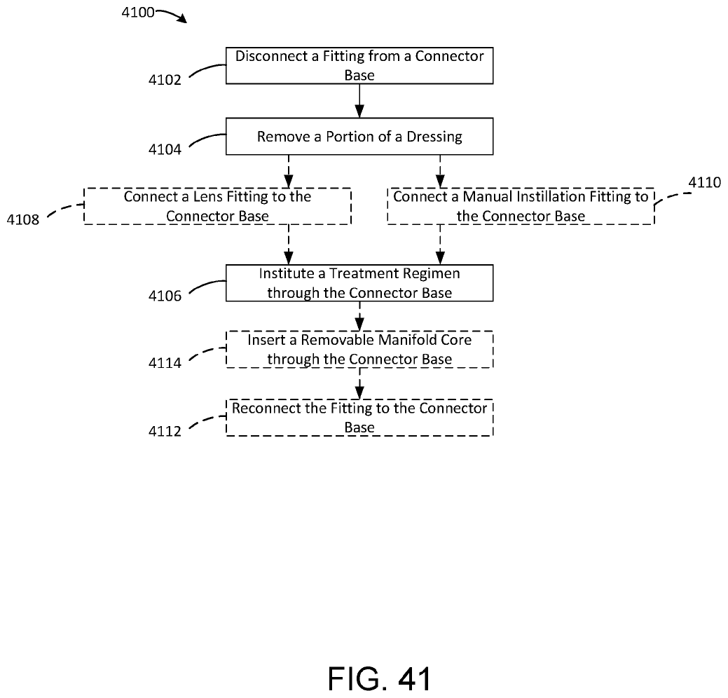

Referring now to FIG. 41, a method of using a NPWT device with a releasable connector 4100 is shown, according to an exemplary embodiment. Method 4100 may be a method of accessing a wound bed through an in-situ negative pressure wound therapy dressing. Method 4100 is implemented with a NPWT device, such as therapy device 102. Method 4100 is implemented with a releasable connector and a fitting. The releasable connector includes a connector base and a fitting. The connector base may be any of connector base 205, connector base 301, connector base 701, connector base 1101, connector base 1501, connector base 1901, connector base 2301, connector base 2701, connector base 3101, and connector base 3501, alone or in combination. The fitting may be any of fitting 218, fitting 302, fitting 702, fitting 1102, fitting 1502, fitting 1902, fitting 2302, fitting 2702, fitting 3102, and fitting 3502, alone or in combination. Method 4100 may also be implemented with a removable manifold core. The removable manifold core may be any of removable manifold core 321, removable manifold core 1128, removable manifold core 1532, removable manifold core 1920, removable manifold core 2318, removable manifold core 2718, removable manifold core 3114, and removable manifold core 3515, alone or in combination.

Method 4100 includes, in block 4102, disconnecting a fitting from a connector base. When the fitting is disconnected from the connector base, an in-situ negative pressure wound therapy dressing may be exposed. Method 4100 then includes, in block 4104, removing a portion of a dressing, such as an in-situ negative pressure wound therapy dressing. Method 4100 then includes, in block 4106, instituting a treatment regimen through the connector base. In some applications, the treatment regimen is conducting a biopsy. In some applications, the treatment regimen is conducting an inspection of a wound bed. In some applications, the treatment regimen is manually instilling a fluid to a wound bed. In some applications, the treatment regimen is obtaining a wound exudate sample. In some applications, the treatment regimen is installing a new tube and/or a new fitting.

Method 4100 may also include, after removing a portion of the dressing and before instituting a treatment regimen through the connector base, in block 4108, coupling a lens fitting to the connector base. The treatment regimen instituted in block 4106 may then be conducting an inspection of the wound bed through the lens fitting.

Method 4100 may also include, after removing a portion of the dressing and before instituting a treatment regimen through the connector base, in block 4110, coupling a manual instillation fitting to the connector base. The treatment regimen instituted in block 4106 may then be manually instilling a fluid to a wound bed.

Method 4100 may also include, after instituting a treatment regimen, in block 4112, reconnecting the fitting to the connector base. This reconnection may form a substantially air-tight seal between the fitting and the connector base. In some applications, the reconnection occurs through a rotation of the fitting relative to the connector base, thereby forming the substantially air-tight seal through rotation of the fitting relative to the connector base. In some applications, the reconnection occurs through a lateral translation of the fitting relative to the connector base, thereby forming the substantially air-tight seal through lateral translation of the fitting relative to the connector base. In some applications, the reconnection occurs through an axial translation of the fitting relative to the connector base, thereby forming the substantially air-tight seal through axial translation of the fitting relative to the connector base.

Method 4100 may also include, after instituting a treatment regimen, in block 4114, inserting a removable manifold core (e.g., a removable and replaceable absorbent core, etc.) through the connector base. Method 4100 may also include block 4112 after block 4414.

Configurations of Releasable Connectors for Use with a NPWT System

The various features of connector base 205, connector base 301, connector base 701, connector base 1101, connector base 1501, connector base 1901, connector base 2301, connector base 2701, connector base 3101, and connector base 3501 described herein are for illustrative purposes only and are not intended be limiting. For example, any features described with relation to any of connector base 205, connector base 301, connector base 701, connector base 1101, connector base 1501, connector base 1901, connector base 2301, connector base 2701, connector base 3101, and connector base 3501 may similarly be implemented on any of the others of connector base 205, connector base 301, connector base 701, connector base 1101, connector base 1501, connector base 1901, connector base 2301, connector base 2701, connector base 3101, and connector base 3501.

Similarly, the various features of fitting 218, fitting 302, fitting 702, fitting 1102, fitting 1502, fitting 1902, fitting 2302, fitting 2702, fitting 3102, and fitting 3502 described herein are for illustrative purposes only and are not intended be limiting. For example, any features described with relation to any of fitting 218, fitting 302, fitting 702, fitting 1102, fitting 1502, fitting 1902, fitting 2302, fitting 2702, fitting 3102, and fitting 3502 may similarly be implemented on any of the others of fitting 218, fitting 302, fitting 702, fitting 1102, fitting 1502, fitting 1902, fitting 2302, fitting 2702, fitting 3102, and fitting 3502.

Connector base 205, connector base 301, connector base 701, connector base 1101, connector base 1501, connector base 1901, connector base 2301, connector base 2701, connector base 3101, connector base 3501, fitting 218, fitting 302, fitting 702, fitting 1102, fitting 1502, fitting 1902, fitting 2302, fitting 2702, fitting 3102, and fitting 3502 may be sterilized. For example, connector base 205, connector base 301, connector base 701, connector base 1101, connector base 1501, connector base 1901, connector base 2301, connector base 2701, connector base 3101, connector base 3501, fitting 218, fitting 302, fitting 702, fitting 1102, fitting 1502, fitting 1902, fitting 2302, fitting 2702, fitting 3102, and fitting 3502 may be sold in a sterile container (e.g., without a dressing, integrated within a dressing, etc.).

Connector base 205, connector base 301, connector base 701, connector base 1101, connector base 1501, connector base 1901, connector base 2301, connector base 2701, connector base 3101, connector base 3501, fitting 218, fitting 302, fitting 702, fitting 1102, fitting 1502, fitting 1902, fitting 2302, fitting 2702, fitting 3102, and fitting 3502 may be reusable, disposable, and/or recyclable.

Dressing 110 and dressing 202 may be foam and/or may include foam dressing layers.

In some embodiments, connector base 205, connector base 301, connector base 701, connector base 1101, connector base 1501, connector base 1901, connector base 2301, connector base 2701, connector base 3101, connector base 3501, fitting 218, fitting 302, fitting 702, fitting 1102, fitting 1502, fitting 1902, fitting 2302, fitting 2702, fitting 3102, and fitting 3502 are constructed from a biocompatible material. Connector base 205, connector base 301, connector base 701, connector base 1101, connector base 1501, connector base 1901, connector base 2301, connector base 2701, connector base 3101, connector base 3501, fitting 218, fitting 302, fitting 702, fitting 1102, fitting 1502, fitting 1902, fitting 2302, fitting 2702, fitting 3102, and fitting 3502 may be constructed from, for example, biocompatible polymer, styrene, polystyrene, and other similar materials. Connector base 205, connector base 301, connector base 701, connector base 1101, connector base 1501, connector base 1901, connector base 2301, connector base 2701, connector base 3101, connector base 3501, fitting 218, fitting 302, fitting 702, fitting 1102, fitting 1502, fitting 1902, fitting 2302, fitting 2702, fitting 3102, and fitting 3502 may be substantially hard and rigid (e.g., compared to a dressing, etc.). For example, connector base 205, connector base 301, connector base 701, connector base 1101, connector base 1501, connector base 1901, connector base 2301, connector base 2701, connector base 3101, connector base 3501, fitting 218, fitting 302, fitting 702, fitting 1102, fitting 1502, fitting 1902, fitting 2302, fitting 2702, fitting 3102, and fitting 3502 may be substantially rigid so as to withstand numerous uses thereof (e.g., numerous coupling and decoupling, numerous removals and insertions of a removable manifold core, etc.).

### Configuration of Exemplary Embodiments

The construction and arrangement of the systems and methods as shown in the various exemplary embodiments are illustrative only. Although only a few embodiments have been described in detail in this disclosure, many modifications are possible (e.g., variations in sizes, dimensions, structures, shapes and proportions of the various elements, values of parameters, mounting arrangements, use of materials, colors, orientations, etc.). For example, the position of elements can be reversed or otherwise varied and the nature or number of discrete elements or positions can be altered or varied. Accordingly, all such modifications are intended to be included within the scope of the present disclosure. The order or sequence of any process or method steps can be varied or re-sequenced according to alternative embodiments. Other substitutions, modifications, changes, and omissions can be made in the design, operating conditions and arrangement of the exemplary embodiments without departing from the scope of the present disclosure.

The present disclosure contemplates methods, systems and program products on any machine-readable media for accomplishing various operations. The embodiments of the present disclosure can be implemented using existing computer processors, or by a special purpose computer processor for an appropriate system, incorporated for this or another purpose, or by a hardwired system. Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. Combinations of the above are also included within the scope of machine-readable media. Machine-executable instructions include, for example, instructions and data which cause a general purpose computer, special purpose computer, or special purpose processing machines to perform a certain function or group of functions.

Although the figures show a specific order of method steps, the order of the steps may differ from what is depicted. Also two or more steps can be performed concurrently or with partial concurrence. Such variation will depend on the software and hardware systems chosen and on designer choice. All such variations are within the scope of the disclosure. Likewise, software implementations could be accomplished with standard programming techniques with rule based logic and other logic to accomplish the various connection steps, processing steps, comparison steps and decision steps.

1. A releasable connector for a negative pressure wound therapy system having a drape and a tube, the releasable connector comprising:
a base configured to be coupled to the drape such that a substantially air-tight seal is formed between the base and the drape, the base comprising a base locking member; and
a tube fitting configured to be coupled to the tube such that a substantially air-tight seal is formed between the tube fitting and the tube, the tube fitting comprising a tube locking member, the tube fitting further configured to be selectively coupled to the base through an interaction between the tube locking member and the base locking member such that a substantially air-tight seal is formed between the tube fitting and the base.

2. The releasable connector of claim 1, wherein the interaction between the tube locking member and the base locking member is a rotation of the tube fitting relative to the base.

3. The releasable connector of claim 1, wherein the interaction between the tube locking member and the base locking member is a lateral translation of the tube fitting relative to the base.

4. The releasable connector of claim 1, wherein the interaction between the tube locking member and the base locking member is an axial translation of the tube fitting relative to the base.

5. The releasable connector of claim 1, further comprising a lens fitting comprising a magnifying lens and a lens fitting locking member configured to be selectively coupled to the base to permit examination of a wound bed.

6. The releasable connector of claim 5, wherein the interaction between the lens fitting locking member and the base locking member is one of:
a rotation of the lens fitting relative to the base;
a lateral translation of the lens fitting relative to the base; or
an axial translation of the lens fitting relative to the base.

7. The releasable connector of claim 5, wherein the base is configured to be coupled to only one of the lens fitting and the tube fitting at any one time.

8. The releasable connector of claim 1, further comprising a removable and replaceable absorbent core accessible through the base.

9. The releasable connector of claim 8, wherein the absorbent core is configured to be removable through the base to permit access to a wound bed.

10. The releasable connector of claim 1, further comprising a manual instillation connection fitting configured to be selectively coupled to the base to permit manual instillation of a fluid to a wound bed or analysis of wound exudate from a wound bed.

11. The releasable connector of claim 10, wherein the base is configured to be coupled to only one of the manual instillation connection fitting and the tube fitting at any one time.

12. A method of accessing a wound bed through an in-situ negative pressure wound therapy dressing, the method comprising:
disconnecting a tube fitting from a base to expose the in-situ negative pressure wound therapy dressing;
removing a portion of the in-situ negative pressure wound therapy dressing through the base; and
instituting a treatment regimen through the base.

13. The method of claim 12, wherein the treatment regimen includes at least one of:
conducting a biopsy;
conducting an inspection of the wound bed;
manually instilling a fluid to the wound bed;
obtaining a wound exudate sample; or
installing a new tube.

14. The method of claim 12, further comprising, after the portion of the in-situ negative pressure wound therapy dressing has been removed through the base and before the treatment regimen has been instituted through the base, coupling a lens to the base; wherein the treatment regimen comprises conducting an inspection of a wound bed through the lens.

15. The method of claim 12, further comprising, after the portion of the in-situ negative pressure wound therapy dressing has been removed through the base and after the treatment regimen has been instituted through the base, reconnecting the tube fitting to the base to cover the in-situ negative pressure wound therapy dressing such that a substantially air-tight seal is formed between the tube fitting and the base.

16-18. (canceled)

19. The method of claim 12, further comprising, after the treatment regimen has been instituted, inserting a removable and replaceable absorbent core through the base.

20. The method of claim 19, further comprising, after the removable and replaceable absorbent core has been inserted through the base, reconnecting the tube fitting to the base to cover the in-situ negative pressure wound therapy dressing.

21. (canceled)

22. A negative pressure wound therapy system comprising:
a dressing comprising a foam material;
a drape having an opening overlaying the foam material;
a connector base coupled to the drape about the opening, the connector base having one of a receptacle and a projection;
a tube fitting having the other of the receptacle and the projection, the tube fitting releasably engageable with the connector base via a coupling interaction between the receptacle and the projection;
a tube coupled to the tube fitting; and
a negative pressure device coupled to the tube.

23. The negative pressure wound therapy system of claim 22, wherein the coupling interaction between the receptacle and the projection creates a substantially air-tight seal between the connector base and the tube fitting.

24. The negative pressure wound therapy system of claim 22, wherein the coupling interaction is one of:
a rotation of the tube fitting relative to the connector base;
a linear translation of the tube fitting relative to the connector base; and
an axial translation of the tube fitting relative to the connector base.

25. The negative pressure wound therapy system of claim 22, further comprising, a removable and replaceable absorbent core configured to be received within the connector base.

26. The negative pressure wound therapy system of claim 22, further comprising a lens fitting comprising a magnifying lens and configured to be selectively coupled to the connector base to facilitate examination of a wound bed.

27. The negative pressure wound therapy system of claim 26, wherein the connector base is configured to be coupled to only one of the lens fitting and the tube fitting at any one time.

28. The negative pressure wound therapy system of claim 22, further comprising a manual instillation connection fitting configured to be selectively coupled to the connector base to permit manual instillation of a fluid to a wound bed or analysis of wound exudate from a wound bed.

29. The negative pressure wound therapy system of claim 28, wherein the connector base is configured to be coupled to only one of the manual instillation connection fitting and the tube fitting at any one time.
